(19) United States
(12) Patent Application Publication
(10) Pub. No.: US 2002/0106690 A1
(43) Pub. Date: Aug. 8, 2002

(54) THE G-PROTEIN B SUBUNIT INTERACTION DOMAIN OF STE20P/PAK FAMILY OF PROTEIN KINASES AND USES THEREOF IN BIOASSAYS

(76) Inventors: EKKEHARD LEBERER, BEACONSFIELD (CA); THOMAS LEEUW, SAUGAU (DE); MALCOLM WHITEWAY, POINTE CLAIRE (CA); DAVID Y. THOMAS, MONTREAL WEST (CA)

Correspondence Address: GOUDREAU GAGE DUBUC, 800 PLACE VICTORIA, SUITE 3400, MONTREAL, QUEBEC, H4Z 1E9, CA

(21) Appl. No.: 09/226,248

(22) Filed: Jan. 7, 1999

(30) Foreign Application Priority Data
Jan. 7, 1998 (CA) 2,219,958

(51) Int. Cl. G01N 33/53

(52) U.S. Cl. 435/7.1

(57) ABSTRACT

The present invention relates generally to signal transduction through G-protein-coupled receptors and more particularly to the interaction between the β subunit of the heterotrimeric G-protein and the Ste20p/PAK family of protein kinases. More particularly, the invention is directed to the identification of the G-protein β subunit interaction domain of Ste20p/PAK family of protein kinases, the Ste20p/PAK interaction domain of G-protein β subunit, to antibodies specific for these interacting domains, the nucleic acid molecules encoding same, to assays, expression vectors, indicator cells, strains, methods and agents which make use of this Ste20p/PAK—G_{β} interaction.

### FIELD OF THE INVENTION

The present invention relates generally to signal transduction in cells. More particularly, the present invention relates to signal transduction through G-protein-coupled receptors and especially to the interaction between β subunits of heterotrimeric G-proteins and the Ste20p/PAK family of protein kinases. The invention also relates to assays, expression vectors, strains, methods and agents which make use of this Ste20p/PAK-G_{β} interaction.

### BACKGROUND OF THE INVENTION

The transmission of numerous extracellular signals through the cell membrane, eventually leading to gene expression modulation, is effected through the interplay of G-protein-coupled receptors (GPCR, one of the most ubiquitous transmembrane receptor families) and a heterotrimeric complex of nucleotide-binding regulatory proteins. This complex, also termed tripartite G-proteins or heterotrimeric G-proteins, is comprised of three subunits termed α, β, and γ. These subunits which can transduce the extracellular signal through the GPCR downstream to different signal transduction pathways are the basis for a wide variety of cell signalling functions involved for example in intercellular communication, response to environmental stimuli such as growth factors, hormones, neurotransmitters, physical parameters (such as light and temperature) and the like. Of importance, the G-protein dependent signalling pathway is conserved in organisms ranging from yeast to man. Due to the structural and functional homologies between the G-proteins in diverse organisms, the yeast Saccharomyces cerevisiae is used as a model system for higher eukaryotic cells and organisms. In fact, numerous factors involved in G-protein signalling have been shown to functionally substitute for the yeast equivalents. The tripartite G-protein complex for example, was shown to be functionally reconstituted using mammalian G_{α} and yeast G_{βγ} (WO 95/21925). In view of the diversity and importance of the signals which induce the G-protein dependent signal transduction pathway, and the importance of the downstream effectors of the G-proteins, the dissection of the interactions taking place in these signal transduction pathways have tremendous fundamental and commercial potential. Furthermore, these interactions represent targets for therapeutic agents. Indeed, the importance of the G-protein-dependent signalling pathway in regulating critical cellular biological functions is demonstrated by the identification of disease conditions which are influenced or determined by mutations in this pathway. For example, the role of GPCRs in disease is reviewed in Coughlin (1994, Curr. Op. Cell. Biol., 6:191-197). Examples of mutations of GPCRs responsible for human diseases have been described (WO 96/41169 and references therein). Moreover, the treatment of a variety of disease conditions is effected through a modulation of the G-protein signalling pathway. For example, agonist analogs of gonadotropin-releasing hormone have been used to treat breast and prostate cancer, endometriosis and non-tumorous ovarian hyperandrogenic syndrome (Pace et al., 1992, Am. Fam. Physician, 44:1777-1782). In view of the critical role played by G-protein signal transduction in cellular homeostasis and disease conditions there remains a need to identify modulators of the G-protein signalling pathways downstream from GPCRs.

The p21-activated protein kinase (PAK) family is a large growing family of regulatory enzymes involved in varied cellular processes ranging from cellular morphogenesis, stress response and apoptosis. The PAK family or Ste20p/PAK family was originally identified based on the property of its kinases to bind to the activated Rho-type p21GTPases Cdc42 and its related protein Rac1. The signature for this family of kinases is a characteristic sequence in the subdomain VIII of the kinase domain (FIG. A; Sells et al., 1997, Trends Cell. Biol., 7:162-167).

The Ste20p/PAK family of protein kinases is divided into three groups or sub-families: (1) the so-called true PAKs which contain an N-terminal p21 binding domain (PBD); (2) the pleckstrin-homology (PH) PAKs which also contain a PH-domain upstream of the PBD; and (3) the GCK sub-family exemplified by the germinal center kinase (GCK), which have a long C-terminal region and lacking a recognizable PBD (FIG. A).

Like Raf, PAKs link GTPases to a protein kinase cascade. However, unlike Raf, for which the activation by Ras can be attributed in large part to a relocalization of the kinase to the plasma membrane, PAK-p21 interaction alone is sufficient for in vitro activation. PAK-Rac and Raf-Ras interactions therefore display both common and different characteristics.

Ste20p kinase, the founding member of the Ste20p/PAK family, shares sequence similarity to protein kinase C, and is required to transmit the pheromone signal from G_{βγ} to downstream components of the signalling pathway (Leberer et al., 1992, EMBO J., 11:4815-4824). Ste20p/PAK has been shown to be a pivotal point between the G-protein-coupled receptors/G_{β}-proteins and the mitogen activated protein kinase (MAP kinase) pathway (Leberer et al., 1997, Curr. Opinion. Genet. & Devel., 7:59-66).

The implication of Ste20p in the activation of a protein kinase cascade prompted the analysis of a similar phenomenon in mammalian cells. Although a definite role for Ste20p/PAKs as major effectors in the stress activated protein kinase cascades (SAPK) has yet to be formally demonstrated, their implication therein has been described (Sells et al., 1997, supra). Indeed, the yeast Ste20p regulated pathways such as mating and filamentous growth share similarities with the JNK/SAPK pathway in mammalian cells which is thought to be activated, at least in part, by a cascade of small G-proteins and homologs of Ste20p (Leberer et al., 1997, supra). As with Ste20p in yeast, PAKs appear to be involved in morphological responses such as membrane ruffling and the formation of focal adhesions which might be functionally equivalent to mating protrusions in yeast (Leberer et al., 1997, supra). Further, the similarity of Ste20p to mammalian p65 PAK (Leberer et al., 1992, supra and U.S. Pat. No. 5,605,825) and of Cdc42p to the mammalian Rho-like guanosine triphosphate Rac1, Cdc42Hs and RhoA, which are known to participate in the activation of the JNK/SAPK signalling cascade and the regulation of actin reorganization in response to extracellular signals, indicates that signal transduction through Ste20p/PAK may be relevant to the understanding of similar signalling mechanisms in organisms ranging from yeast to mammalian cells (Leeuw et al., 1995, Science, 270:1210-1213). The answers obtained using Ste20p in yeast are therefore of importance in the global understanding of Ste20p/PAK implications in various signalling cascades in eukaryotes in general.

Recent examples have shown the importance of the G-protein-coupled receptor-tripartite G-proteins—Ste20p/PAK interactions (Knaus et al., 1995, Science, 269:221-223; Teo et al., 1995, J. Biol. Chem., 270:26690-26697). It has been established that G-protein coupled receptors can regulate PAKs in mammalian cells. Chemoattractants were shown to rapidly stimulate two human PAKs through the activation of heterotrimeric G-proteins leading to the phosphorylation of p47^{phox}, suggesting an implication of G-protein-PAKs in NADPH oxidase regulation, and hence, in inflammatory response of human phagocytic leucocytes. Further, thrombin, which binds to a classical G-protein coupled receptor was shown to activate γ-PAK, a platelet protein kinase displaying significant identity to human p65 PAK, suggesting that PAK may be a part of the thrombin-response signalling complex and platelet function (Teo et al., 1995, supra).

Like PAKs, a number of GCK-like PAK members (referred as group (3) above) activate kinase cascades such as the aforementioned Jun N-terminal kinase (JNK) cascade, the stress activated protein kinase (SAPK cascade) and the mitogen activated protein kinase (MAPK cascade). Although sequence similarities between GCK and PAK family members seem limited primarily to the kinase domain, the identification of the p21 binding motifs in the rat homolog of GCK, raises the possibility that other GCK-PAK-subfamily members might have non-recognized PBDs (Sells et al., 1997, supra).

The recent identification of HIV's essential protein Nef as associating with and activating at least one PAK-like kinase further indicates that PAKs and homologs thereof have the potential to play an important role in animal diseases and in human diseases in particular (Sells et al., 1997, supra).

The mating-pheromone response in yeast provides a genetically tractable system to study structure/function relationships of the G-protein-Ste20p signal transduction pathway and related pathways in vivo. In view of the high degree of functional and structural homologies between the G-proteins and downstream effectors such as the Ste20p/PAK proteins, the yeast system has the potential to provide critical insights into signal transduction pathways in higher eukaryotes (Lebereretal., 1992, EMBO J., 11:4805-4813).

The yeast mating-response MAP kinase cascade consists of Ste11p (a MAP or extracellular signal regulated kinase kinase (MEK) kinase homolog), Ste7p (a MEK homolog) and the partially redundant MAP kinase homologs Fus3p and Kss1p (Leberer et al., 1997, supra). Activation of this cascade through binding of pheromones to G-protein coupled receptors induces cellular processes which are typical of differentiating cells, including growth arrest in G₁ of the cell cycle, differential gene expression, and polarized morphogenesis which leads to the formation of mating-specific projections (Leberer et al., 1997, supra). G_{β}-mediated activation of this cascade involves Ste20p (a MEK kinase kinase) and the MAP kinase scaffolding protein Ste5p (Leberer et al., 1997, supra). PAKs, a subgroup of mammalian Ste20p homologs, can be activated by either the small G-proteins Cdc42 and Rac or by heterotrimeric G-proteins in various signalling pathways (Sells et al., 1997, supra). The Cdc42p binding domain of Ste20p has been shown to be dispensable for pheromone signalling in yeast suggesting that activation of Ste20p in response to pheromone occurs in a manner independent of Cdc42p (Peter et al., 1996, EMBO J., 15:7046-7059; Leberer et al., 1997, supra).

The importance of the Ste20p/PAK family of protein kinases is supported by the significant functional and structural conservation thereof throughout evolution. The recent discovery that certain GCK/PAK subfamily members may also couple with GTPases raises the possibility that PAKs in general may mediate GTPase functions. In view of the critical and often essential roles of such Ste20p/PAK interactions in fundamental and diverse cellular processes, and the conservation of the structure/function relationship of PAKs throughout evolution, there is a tremendous need in dissecting and understanding the molecular determinants involved in Ste20p/PAK-G-protein interactions. Such dissections and understandings might shed a light on the possibility that differential regulation by heterotrimeric and small G-proteins may contribute to Ste20p/PAK specificity on the downstream MAP kinase module, and may explain how the same protein kinase module may regulate different developmental pathways within the same cell.

The present invention seeks to meet these and other needs.

The present description refers to a number of documents, the content of which is herein incorporated by reference.

### SUMMARY OF THE INVENTION

The invention concerns the identification of the domains implicated in the Ste20p/PAK—Ste4p/G_{β} interaction. More particularly, the invention relates to the G_{β} interaction domain of Ste20p and homologs thereof.

The present invention relates to the identification of the molecular determinants of Ste4p/G_{β} interaction in Ste20p/PAK. The invention further relates to the identification of a Ste20p/PAK interaction domain in Ste4p/G_{β}.

Also, the invention relates to a characterization of the molecular determinant of a Ste20p/PAK interaction domain in Ste4p/G_{β}.

The present invention further relates to isolated polypeptides containing a Ste4p/G_{β} interaction domain of Ste20p/PAK.

As well, it relates to isolated polypeptides containing a Ste20p/PAK interaction domain of Ste4p/G_{β}.

Further, the invention relates to epitope-binding portions of the polypeptides of the present invention.

In a preferred embodiment, the Ste4p/G_{β} interaction domain of Ste20p/PAK comprises the amino acid sequence as set forth in the consensus sequence SSLφPLI_{V}Xφφβ and as set forth in SEQ. ID. NO.: ID. NO.:27. In a particular embodiment, the Ste20p/G_{β} interaction domain of Ste20p/PAK comprises an amino acid sequence in accordance with the above consensus sequence. Examples of such sequences include sequences as set forth in SEQ. ID. NO.: ID. NOs.:1, 2, 4-11 or derivatives or fragments thereof. Ste20p/G_{β} interaction domains having a sequence with significant homology to the consensus are also provided for example in SEQ. ID. NO.: ID. NO.:3, 12 and 13 or derivatives or fragments thereof.

In another embodiment, a Ste4p/G_{β} interaction domain of Ste20p/PAK comprises a more divergent amino acid sequence as set forth in SEQ. ID. NO.: ID. NOs.:14-20 or derivatives or fragments thereof, as compared to the above-listed consensus sequence.

In yet another preferred embodiment, the Ste20p/PAK interaction domain of Ste4p/G_{β} comprises the amino acid sequence as set forth in SEQ. ID. NO.: ID. NOs.:21-25 or derivatives or fragments thereof.

The invention in addition relates to nucleic acid sequences encoding a Ste4p/G_{β} interaction domain of Ste20p/PAK and to nucleic acid sequences encoding a Ste20p/PAK interaction domain of Ste4p/G_{β}. In one particular embodiment, the nucleic acid sequences encoding a Ste4p/G_{β} domain of Ste20p/PAK encode the amino acid sequence as setforth in one of SEQ. ID. NO.: ID. NOs.:1-13 or functional derivatives thereof, in SEQ. ID. NO.: ID. NOs.: 14-20 or to a nucleic acid sequence which hybridizes thereto under high stringent conditions or is at least 90% identical to such nucleic acid sequences encoding the Ste4p/G_{β} binding domain of the present invention.

In another embodiment, the nucleic acid sequence encoding the Ste20p/PAK interaction domain of Ste4p/G_{β} encodes the amino acid sequence as set forth in SEQ. ID. NOs.:21-26 or derivatives or fragments thereof or to a nucleic acid sequence which hybridizes thereto under high stringent conditions or is at least 90% identical to nucleic acid sequences encoding the Ste20p/PAK interaction domain of the present invention. In a preferred embodiment, the nucleic acid sequences of the present invention are as set forth in SEQ. ID. NOs.:28 and 30, derivatives or fragments thereof, or nucleic acid sequences which hybridize thereto under stringent conditions or are at least 90% identical thereto.

The present invention also seeks to provide a recombinant nucleic acid molecule comprising an isolated nucleic acid of the present invention operably linked to a promoter element; cells containing same, and vectors and host cells harboring such vectors for expressing the polypeptides of the invention.

The present invention also seeks to provide antibodies directed to the polypeptides or epitope bearing portions thereof as well as to hybridomas producing monoclonal antibodies directed against such polypeptides.

The invention further seeks to provide methods and compositions to screen for compounds having the ability to modulate a signal transduction pathway through their modulation of the Ste20p/PAK—Ste4p/G_{β} interaction. In one aspect of the present invention, the compound inhibits the Ste20p/PAK—Ste4p/G_{β} interaction and uncouples the G-protein receptor from downstream cascades. In another aspect, the agent enhances the Ste20p/PAK—Ste4p/G_{β} interaction, thereby inducing the activation of a downstream signal transduction cascade. In a particular aspect of the present invention, the abilities of a compound(s) to modulate a signal transduction pathway through their modulation of the Ste20p/PAK—Ste4p/G_{β} interaction is assessed by measuring effects on cellular metabolism. In a particular embodiment, this assessment is made through the use of yeast cells as indicator cells and the effect of the test compound(s) observed through the mating ability of the yeast cells. In another particular embodiment, this assessment is made through in vitro means well known to the person of ordinary skill. Non limiting examples of such in vitro means include enzyme-linked immunosorbent assays (ELISA) or other immunological assays, filter binding assays, scintillation proximity assays and the like. Once identified such Ste20p/PAK—Ste4p/G_{β} modulating agents can be used as lead compounds to search for drugs, that can modulate a particular signal transduction pathway.

The present invention is also directed to pharmaceutical compositions for controlling diseases which are dependent on the interaction between Ste20p/PAK and Ste4p/G_{β}. As well, the invention relates to the administration of such compositions to an animal suffering from a disease which is dependent on the aforementioned interaction.

Accordingly, the present invention also seeks to provide an assay kit for screening and identifying compounds which modulate the Ste20p/PAK—Ste4p/G_{β} interaction wherein the kit contains a first polypeptide comprising a Ste4p/G_{β} interaction domain of Ste20p/PAK and a second polypeptide comprising a Ste20p/PAK interaction domain of Ste4p/G_{β}, and wherein the interaction of the interacting domains is assayable.

The present invention in addition seeks to provide a method for screening and identifying compounds which modulate the Ste20p/PAK—Ste4p/G_{β} interaction, comprising the step of incubating a compound in admixture with a substantially purified first and second polypeptide, wherein the first polypeptide comprises a Ste4p/G_{β} interaction domain of Ste20p/PAK and the second polypeptide comprises a Ste20p/PAK interaction domain of Ste4p/G_{β}, and determining the extent to which the compound modulates the interaction between the two polypeptides as compared to a control incubation in the absence of the compound.

In a particular aspect, the present invention seeks to provide a method of controlling diseases, dependent on an interaction of Ste20p/PAK and Ste4p/G_{β} in an animal such as a mammal and to pharmaceutical compositions therefor.

In addition, the present invention seeks to provide a non-human organism containing the nucleic acid molecule encoding an interaction domain of the present invention. The present invention also seeks to provide a non-human organism containing a knock-out of an interaction domain of the present invention.

The polypeptides and nucleic acid sequences of the present invention have utility in designing in vitro and in vivo experimental models. Such experimental models enable the screening of large collections of synthetic, semi-synthetic, or natural compounds for therapeutic use in Ste20p/PAK—Ste4p/G_{β}-dependent diseases or applications. The present invention also enables the identification of signalling pathways converging at the Ste20p/PAK—G_{β}Ste4p interaction.

The applicant is the first to demonstrate a direct interaction between Ste20p/PAK and G/Ste4p. Before the present invention, it was not clear whether Ste20p PAK and G_{β} interacted. In addition, the applicant is the first to identify the domains involved in the interaction of Ste20p/PAK with Ste4p/G_{β}, of relevance to the understanding of signal transduction in all eukaryotic organisms.

In accordance with the present invention, there is therefore provided polypeptidic regions involved in the interaction of Ste20p/PAK and Ste4p/G_{β}. As well there is provided nucleic acid molecules encoding such interacting domains. Further, there is provided fusion proteins comprising the interaction domains of the present invention, nucleic acid molecules encoding same and cells harboring those nucleic acid molecules.

In accordance with the present invention, there is also provided, assays and methods for the identification of compounds which modulate the Ste20p/PAK—Ste4p/G_{β} interaction.

In accordance with the present invention, there is additionally provided methods of treatment and uses of compounds which modulate Ste20p/PAK—Ste4p/G_{β} interaction as well as pharmaceutical compositions containing same.

Furthermore, in accordance with the present invention, there is provided, a composition of matter comprising: an isolated Ste4p/G_{β}-binding polypeptide or fragment thereof wherein the isolated Ste4p/G_{β}-binding polypeptide is a Ste20p/PAK polypeptide which directly binds to a Ste4p/G_{β} polypeptide or fragment thereof; and an isolated Ste20p/PAK-binding polypeptide or fragment thereof, wherein the isolated Ste20p/PAK polypeptide is a Ste4p/G_{β} polypeptide which directly binds to a Ste20p/PAK polypeptide or fragment thereof. In addition, in accordance with the present invention, there is also provided, a composition of matter comprising an isolated nucleic acid molecule comprising a nucleic acid sequence which encodes a Ste4p/G_{β}-binding domain of Ste20p/PAK and an isolated nucleic acid molecule comprising a nucleic acid sequence which encodes a Ste20p/PAK-binding domain of Ste4p/G_{β}.

It shall also be understood, that since there is significant homology between the different members of the Ste20p/PAK family members and between the evolutionary divergent Ste4p/G_{β} sequences (see below), that the person of ordinary skill, will be able to adapt the teachings of the present invention in a variety of ways, with amino acid and nucleic acid sequences from different animals and organisms.

### BRIEF DESCRIPTION OF THE DRAWINGS

Having thus generally described the invention, reference will now be made to the accompanying drawings, showing by way of illustration a preferred embodiment thereof, and in which:

FIG. A (prior art) shows a structural comparison of the extended p21-activated Ste20p/PAK family of protein kinases (Sells et al., 1997, Trends in Cell Biol., 7:162-167);

FIG. 1 shows the association of Ste4p with Ste20p and Ste5p in yeast cells. (A) Time course of pheromone-induced Ste20p binding to HA-Ste4p. HA-Ste4p expressed from the STE4 promoter in cells deleted for STE4 was immunoprecipitated after treatment with 1 pM a-factor. Relative amounts of Ste20p and HA-Ste4p were determined in Western blots (see example in upper and middle panels) and quantified densitometrically (mean values ±SD, n=3) (lower panel). (B) Association of Ste5p with HA-Ste4p expressed from the STE4 promoter in cells deleted for STE4 . HA-Ste4p immunoprecipitates from exponentially growing (−) and pheromone-treated (90 minutes) (+) yeast cells were analyzed with antibodies to Ste5p (upper panel) or HA-Ste4p (lower panel). (C) Overexpression of Ste4p leads to binding to Ste20p. HA-Ste4p was overexpressed from the GAL1 promoter in cells deleted for STE20 (lane 1) or STE4 (lanes 2 and 3). HA-Ste4p expression was suppressed in glucose-containing medium in cells deleted for STE4 (lane 4). Immunoprecipitates obtained with antibodies to Ste20p (lanes 1 and 2) or the HA-epitope (lanes 3 and 4) were analyzed for the presence of Ste20p (upper panel) and HA-Ste4p (lower panel). (D) Coimmunoprecipitation of HA-Ste4p and Ste20p truncation mutants. HA-Ste4p and Ste20p⁴⁹⁵⁻⁸⁸⁸ (lanes 1 and 3) or Ste20p⁴⁹⁵⁻⁸⁷⁷ (lanes 2 and 4) truncation mutants were overexpressed from the GAL1 promoter in cells deleted for STE20. HA-Ste4p (lanes 1 and 2) and Ste20p (lanes 3 and 4) immunoprecipitates were analyzed for the presence of Ste20p (upper panel) and HA-Ste4p (lower panel) by Western blot analyses. Multiple bands of HA-Ste4p and Ste20p represent phosphorylated forms as indicated by phosphatase treatment (data not shown).

FIG. 2 shows the In vitro-G_{β} binding assays. (A) Ste4p binds to a sequence carboxyl-terminal to the kinase domain of Ste20p. GST fusions with full length Ste20p (Leberer et al., 1997, supra) (GST-Ste20p FL) and the indicated Ste20p fragments were incubated with in vitro-translated ³⁵S-Ste4p in the presence (left, right and (+) in middle panels) or in the absence ((−), middle panel) of in vitro-translated HA-Ste18p. GST fusion proteins were detected by Western blot analyses with antibodies to GST (upper panels). ³⁵S-Ste4p was detected by autoradiography (lower panels). The presence of HA-Ste18p was confirmed by Western blot analyses (data not shown). (B) Summary of the interactions between Ste20p fragments and Ste4p. The interactions were determined by either in vitro binding assays (a) or coimmunoprecipitations from yeast extracts (b). Conserved residues are underlined in multiple alignments of carboxyl-terminal sequences of Ste20p (Leberer et al., 1992, supra), mouse mPAK3 (Bagrodia et al., supra), rat PAK (Manser et al., 1994, Nature, 367:40-46) and yeast Cla4p (Cvrckova et al., 1995, Genes Dev., 9:1817-1830), and human PAK, CBD, Cdc42p binding domain. (C) Interactions of ³⁵S-Ste4p with mouse mPAK3 and yeast Cla4p. GST and amino-terminal fusions of GST with Ste20p, mouse mPAK3 and Cla4p were incubated with in vitro-translated ³⁵S-Ste4p in the presence (+) or absence (−) of in vitro-translated HA-Ste18p. Analyses of proteins were performed as described in (A). Relative amounts of ³⁵S-Ste4p were normalized for relative levels of full length GST fusion proteins containing the intact carboxyl-terminal Ste4p binding site. Data are given as percent of the amount of ³⁵S-Ste4p bound to GST-Ste20p (mean values ±SD, n>3).

FIG. 3 shows the mutational analyses of the association of Ste4p with Ste20p. Interaction of Ste4p mutants with Ste20p and Ste5p. Fusions of GST with Ste20p and Ste5p were incubated in the presence of in vitro-translated HA-Ste18p with wild-type ³⁵S-Ste4p or the indicated dominant-negative ³⁵S-Ste4p mutants (Leberer et al., 1992, supra). Relative amounts of the GST fusion proteins and of ³⁵S-Ste4p were quantified by densitometric evaluation of Western blots and autoradiographs, respectively. Data are given as percentage of binding of wild-type ³⁵S-Ste4p (mean values ±SD, n≧3).

FIG. 4 shows a model for the role of Ste20p in the activation of the pheromone response pathway.

FIG. 5 shows multiple alignments of the G_{β}-binding sequence of Ste20p with the homologous regions of related protein kinases of the Ste20p/PAK family. All accession numbers are from the Swiss Prot and PIR or GeneBank (in parentheses) databases. Numbers to the left of the first residue from each sequence indicates the position of this residue in the protein sequence (where 1 is the initiator Met). Number to the right depicts the position of the carboxyl terminal residue. Numbers in parathesis are from incomplete sequences. The consensus sequence for the G_{β}-binding motif is show below (where φ is either A, I, L, M, S, or T, and β is a basic residue). Sc, Saccharomyces cerevisiae; Ca, Candida albicans; Sp, Schizosaccharomyces pombe; Hs, Homo sapiens; Dm, Drosophila melanogaster; Xen, Xenopus; Ce, Caenorabditis elegans; Dd, Dictyostelium discoidium; Ac, Acantamoeba.

FIG. 6 shows multiple alignments of yeast Ste4p with mammalian Gβ subunits (Hgbb1, human Gβ1; Hggb2, human Gβ2; Hggb3, human Gβ3; Mgbb4, mouse Gβ4; Mgbb5, mouse Gβ5). The numbers in parentheses are the Swiss Prot accession numbers.

Other objects, advantages and features of the present invention will become more apparent upon reading of the following non-restrictive description of preferred embodiments with reference to the accompanying drawings which are exemplary and should not be interpreted as limiting the scope of the present invention.

### DESCRIPTION OF THE PREFERRED EMBODIMENT

The domains involved in the Ste20p/PAK—Ste4p/G_{β} interaction are herein provided. Isolated polynucleotides and oligonucleotides encoding the Ste20p/PAK—Ste4p/G_{β} interaction domains are provided by the present invention. Isolated proteins encoded by these polynucleotides and oligonucleotides are also provided. Examples of amino acid sequences in accordance with the present invention include SEQ. ID. Nos.:1-27, 29, and 31. Examples of nucleic acid sequences in accordance with the present invention and from which fragments and derivatives thereof can be obtained include SEQ. ID. Nos.:28 and 30.

Certain aspects of the present invention also include nucleic acid sequences which are homologous to the nucleic acid sequences of the present invention.

In another embodiment of the invention, the amino acid sequences of the present invention provide sequences for obtaining polyclonal or monoclonal antibodies, chimeric antibodies, humanized antibodies and the like which are specific for the Ste20p/PAK—Ste4p/G_{β} interaction domain.

Alternatively, in another embodiment the present invention provides a simple, rapid high-throughput functional bioassay for identifying compounds that modulate the Ste20p/PAK—Ste4p/G_{β} interaction. These compounds can act either as agonists or antagonists of Ste20p/PAK—Ste4p/G_{β} interaction and signalling functions. In one embodiment, the assay is an “in vivo” experimental model based on the incubation of indicator cells with test compounds and the identification of the test compound as agonist or antagonist of Ste20p/PAK—Ste4p/G_{β} interaction. Alternatively, it is based on the use of an “in vitro” experimental model such as an enzymatic assay, binding assay and the like (i.e. examples 8 and 9). Compounds can be tested individually or in pools or libraries. The term “antagonist” refers to a compound which inhibits the interaction between Ste20p/PAK and Ste4p/G_{β}, thereby uncoupling signal transduction through G-proteins. Alternatively, the term “agonist” refers to a compound that stimulates such a signal transduction by promoting Ste20p/PAK—Ste4p/G_{β} interaction. The term “modulator” is used herein to refer to a compound or a mixture or pool thereof which positively or negatively affect the Ste20p/PAK—Ste4p/G_{β} interaction.

As used herein, the terms “interaction domains” and “binding domains” are used interchangeably.

As used herein the recitation “indicator cells” refers to cells that express an interaction domain of a Ste4p/G_{β}—Ste20p/PAK and a Ste20p/PAK interaction domain of Ste4p/G_{β}, and wherein an interaction between these domains is coupled to an identifiable or selectable phenotype or characteristic such that it provides an assessment of the interaction between the domains. Such indicator cells can be used in the screening assays of the present invention. In a preferred embodiment, the indicator cells have been engineered so as to replace at least one of the endogenous Ste20p/PAK and Ste4p/G_{β} interacting domains of Ste4p/G_{β} and Ste20p/PAK respectively, by a chosen derivative, fragment, homolog, or mutant thereof. Alternatively, the indicator cells are engineered so as to inhibit the expression of at least one of the aforementioned endogenous interacting domains. The cells can be yeast cells or higher eukaryotic cells such as mammalian cells (WO 96141169). Preferably, the indicator cells are yeast cells. Non-limiting examples of such cells and vectors are exemplified herein below (i.e. examples 7 and 11). In one particular embodiment, an indicator cell of the present invention which is wild type with respect to mating can be used to test a compound or a library thereof in order to identify same which affect mating. In another embodiment, the indicator cell can be a yeast cell harboring vectors enabling the use of the two hybrid system technology as well known in the art (Ausubel et al. 1994, supra). In one embodiment, a reporter gene encoding selectable marker can be operably linked to a control element such that expression of the selectable marker is dependent on the interaction of the Ste20p/PAK—Ste4p/G_{β} interacting domains. Such an indicator cell could be used to rapidly screen at high-throughput a vast array of test compounds. In a particular embodiment, the reporter gene is luciferase, β-Gal or green fluorescent protein. It will be understood that the indicator cell, polypeptides and nucleic acids of the present invention can be engineered to be particularly suited for the expression of heterologous Ste20p/PAK and/or Ste4p/G_{β} proteins (WO 95/21925).

As exemplified herein below in one embodiment, at least one of a Ste20p/PAK and Ste4p/G_{β} interaction domain of the present invention may be provided as a fusion protein. The design of constructs therefor and the expression and production of fusion protein are well known in the art (Sambrook et al., 1989, in Molecular Cloning—A Laboratory Manual, Cold Spring Harbor Laboratories, and Ausubel et al., 1994, Current Protocols in Molecular Biology, Wiley, N.Y.). In certain embodiments, it might be beneficial to introduce a protease cleavage site between the two polypeptide sequences which have been fused. Non-limiting examples of such fusion proteins include a hemagglutinin—Ste4p/G_{β} fusion protein and a Ste20p-GST fusion. In certain embodiments, it might be beneficial to fuse the interaction domains of the present invention to signal peptide sequences enabling a secretion of the fusion protein from the host cell. Signal peptides from diverse organisms are well known in the art. Bacterial OmpA and yeast Suc2 are two non-limiting examples of proteins containing signal sequences.

As used herein, the term “compound” is used broadly to refer to natural, synthetic or semi-synthetic compounds. The term “compound” therefore denotes for examples macromolecules, cell or tissue extracts (from plants or animals). Non-limiting examples of compounds include nucleic acid molecules, peptides, antibodies, carbohydrates and pharmaceutical agents. The agents can be selected and screened by a variety of means including random screening, rational selection and by rational design using for example protein or ligand modelling methods such as computer modelling. The terms “rationally selected” or “rationally designed” are meant to define compounds which have been chosen based on the configuration of the interaction domains of the present invention. As will be understood by the person of ordinary skill, macromolecules having non-naturally occurring modifications are also within the scope of the term “compound”. For example, peptidomimetics, well known in the pharmaceutical industry and generally referred to as peptide analogs can be generated by modelling as mentioned above. Similarly, in a preferred embodiment, the polypeptides of the present invention are modified to enhance their stability. It should be understood that in most cases this modification should not alter the biological activity of the interaction domain. The compounds identified in accordance with the teachings of the present invention have a therapeutic value for the treatment of diseases or conditions which are dependent on Ste20p/PAK—Ste4p/G_{β} interaction. Such diseases or conditions could include proliferative diseases, inflammatory diseases, apoptosis and the like.

As used herein, the term “selectable marker” is used broadly to refer to markers which confer an identifiable trait to the indicator cell. Non-limiting example of selectable markers include markers affecting viability, metabolism, proliferation, morphology and the like.

As used herein, agonists and antagonists of Ste20p/PAK—Ste4p/G_{β} interaction also include potentiators of known compounds with such agonist or antagonist properties. In one embodiment, agonists can be detected by contacting the indicator cell with a compound or mixture or library of compounds for a fixed period of time. The level of gene expression (e.g. the level of luciferase produced) within the treated cells is then determined. The expression level can be compared to that of the reporter gene in the absence of the compound(s). The difference between the levels of gene expression indicates whether the compound(s) of interest agonize the aforementioned interaction. The magnitude of the level of reporter gene product expressed (treated vs. untreated cells) provides a relative indication of the strength of that compound(s) as an agonist. Alternatively, such an indicator cell can be used to identify antagonists.

For example, the test compound or compounds are incubated with the host cell in conjunction with one or more known agonists held at a fixed concentration. An indication and relative strength of the antagonistic properties of the compound(s) can be provided by comparing the level of gene expression in the indicator cell in the presence of the known agonist, in the absence of test compounds vs in the presence thereof.

It shall be understood that the “in vivo” experimental model can also be used to carry out an “in vitro” assay. For example, cellular extracts from the indicator cells can be prepared and used in one of the aforementioned “in vitro” tests (i.e. example 11). Numerous in vitro methods to detect and/or quantify the interaction between two interacting polypeptides are known to the person of ordinary skill. For example, antibodies can be used for this purpose. The conditions and the type of assay can be adapted by the person of ordinary skill as a function of the desired type of information required, the format of the assay, the detection method and the type and nature of the antibody used. Non limiting examples of commonly known immunological assays which can be used to assess the interaction between Ste20p/PAK and Ste4p/G_{β} include radioimmunoassays, ELISA, immunofluorescence-type assays and the like. Immunological assays which can be used in the context of the present invention are described for example in Harlow et al., 1988 (in: Antibody—A Laboratory Manual, CSH Laboratories). As well different type of binding assays, for example direct or indirect, or competitive binding assays can be used. Scintillation proximity-type assays are other non limiting examples of assays which can be used to identify compounds which modulate the Ste20p/PAK—Ste4p/G_{β} interaction.

For certainty, as used herein “Ste20p/PAK” and “Ste4p/G_{β}” refer herein to members of the Ste20p/PAK family of protein kinases and to homologs of “G_{β}”, respectively. Thus, any Ste20p/PAK or any Ste4p/G_{β} family member with the proviso that it comprises the interaction domains of the present invention or nucleic acid sequences encoding same can be used to practice the present invention. For certainty, the sequences and polypeptides useful to practice the invention include without being limited thereto mutants, homologs, subtypes, alleles and the like. It shall be understood that generally, the sequences of the present invention should encode a functional (albeit defective) interaction domain. It will be clear to the person of ordinary skill that whether an interaction domain of the present invention, variant, derivative, or fragment thereof retains its function in binding to its partner can be readily determined by using the teachings and assays of the present invention and the general teachings of the art. As exemplified herein below, the interaction domains of the present invention can be modified, for example by in vitro mutagenesis, to dissect the structure-function relationship thereof and permit a better design and identification of modulating compounds. However, some derivative or analogs having lost their biological function of interacting with their respective interaction partner (Ste20p/PAK or Ste4p/G_{β}) may still find utility, for example for raising antibodies. Such analogs or derivatives could be used for example to raise antibodies to the interaction domains of the present invention. These antibodies could be used for detection or purification purposes. In addition, these antibodies could also act as competitive or non-competitive inhibitor and be found to be modulators of Ste20p/PAK—Ste4p/G_{β} interaction.

A consensus sequence of the Ste4p/G_{β} interaction domain is herein provided. It shall be clear that a 100% identity to this consensus sequence is not necessary to provide functionality to Ste20p/PAK (binding to Ste4p/G_{β}) since for example (and as described below), a serine to alanine substitution at the first aa position thereof (DPak; SEQ. ID. NO.: ID. NO.:12) retains the biological function. The same can be said of SEQ. ID. NO.: ID. NO.:3, since Shk1 of S.Pombe complements a Ste20 gene disruption. More divergent amino acid sequences, as exemplified for example by SEQ. ID. NO.: ID. NO.:17 does not bind, however. Thus, more divergent amino acid sequence such as SEQ. ID. NO.: ID. NOs.:14-20 and especially SEQ. ID. NO.: ID. NOs.:17-20 can be used to identify compounds and/or molecular determinants of the sequence which can stimulate the Ste4p/G_{β} interaction.

Nucleotide sequences are presented herein by single strand, in the 5′ to 3′ direction, from left to right, using the one letter nucleotide symbols as commonly used in the art and in accordance with the recommendations of the IUPAC-IUB Biochemical Nomenclature Commission.

The present description refers to a number of routinely used recombinant DNA (rDNA) technology terms. Nevertheless, definitions of selected examples of such rDNA terms are provided for clarity and consistency.

As used herein, “isolated nucleic acid molecule”, refers to a polymer of nucleotides. Non-limiting examples thereof include DNA and RNA molecules purified from their natural environment.

The term “recombinant DNA” as known in the art refers to a DNA molecule resulting from the joining of DNA segments. This is often referred to as genetic engineering.

The term “DNA segment”, is used herein, to refer to a DNA molecule comprising a linear stretch or sequence of nucleotides. This sequence when read in accordance with the genetic code, can encode a linear stretch or sequence of amino acids which can be referred to as a polypeptide, protein, protein fragment and the like.

The nucleic acid (i.e. DNA or RNA) for practicing the present invention may be obtained according to well known methods.

A host cell or indicator cell has been “transfected” by exogenous or heterologous DNA (e.g. a DNA construct) when such DNA has been introduced inside the cell. The transfecting DNA may or may not be integrated (covalently linked) into the genome of the cell. In prokaryotes, yeast, and mammalian cells for example, the transfecting DNA may be maintained on an episome such as a plasmid. With respect to eukaryotic cells, a stably transfected cell is one in which the transfecting DNA has become integrated into the genome so that it is inherited by daughter cells upon replication. The stability of the integrated DNA can be demonstrated by the establishment of cell lines or clones comprised of a population of daughter cells containing the transfecting DNA. Transfection methods are well known in the art (Sambrook et al., 1989, supra; Ausubel et al., 1994, supra).

“Nucleic acid hybridization” refers generally to the hybridization of two single-stranded nucleic acid molecules having complementary base sequences, which under appropriate conditions will form a thermodynamically favored double-stranded structure. Examples of hybridization conditions can be found in the two laboratory manuals referred above (Sambrook et al., 1989, supra, and Ausubel et al., 1994, supra) and are commonly known in the art. In the case of a hybridization to a nitrocellulose filter, as for example in the well known Southern blotting procedure, a nitrocellulose filter can be incubated overnight at 65° C. with a labeled probe in a solution containing 50% formamide, high salt (5× SSC or 5× SSPE), 5× Denhardt's solution, 1% SDS, and 100 μg/ml denatured carrier DNA (i.e. salmon sperm DNA). The non-specifically binding probe can then be washed off the filter by several washes in 0.2× SSC/0.1% SDS at a temperature which is selected in view of the desired stringency: room temperature (low stringency), 42° C. (moderate stringency) or 65° C. (high stringency). The selected temperature is based on the melting temperature (Tm) of the DNA hybrid. Of course, RNA-DNA hybrids can also be formed and detected. In such cases, the conditions of hybridization and washing can be adapted according to well known methods by the person of ordinary skill. Stringent conditions will be preferably used (Sambrook et al.,1989, supra).

As used herein, the term “gene” is well known in the art and relates to a nucleic acid sequence defining a single protein or polypeptide. A “structural gene” defines a DNA sequence which is transcribed into RNA and translated into a protein having a specific amino acid sequence thereby giving rise the a specific polypeptide or protein.

A “heterologous” (i.e. a heterologous gene) region of a DNA molecule is a subsegment segment of DNA within a larger segment that is not found in association therewith in nature. The term “heterologous” can be similarly used to define two polypeptidic segments not joined together in nature. Non-limiting examples of heterologous genes include reporter genes such as luciferase, chloramphenicol acetyl transferase, β-galactosidase, and the like which can be juxtaposed or joined to heterologous control regions or to heterologous polypeptides.

The term “vector” is commonly known in the art and defines a plasmid DNA, phage DNA, viral DNA and the like, which can serve as a DNA vehicle into which DNA of the present invention can be cloned. Numerous types of vectors exist and are well known in the art.

The term “expression” defines the process by which a structural gene is transcribed into mRNA (transcription), the mRNA is then being translated (translation) into one polypeptide (or protein) or more.

The terminology “expression vector” defines a vector or vehicle as described above but designed to enable the expression of an inserted sequence following transformation into a host. The cloned gene (inserted sequence) is usually placed under the control of control element sequences such as promoter sequences. The placing of a cloned gene under such control sequences is often referred to as being operably linked to control elements or sequences.

Expression control sequences will vary depending on whether the vector is designed to express the operably linked gene in a prokaryotic or eukaryotic host or both (shuttle vectors) and can additionally contain transcriptional elements such as enhancer elements, termination sequences, tissue-specificity elements, and/or translational initiation and termination sites.

As used herein, the designation “functional derivative” denotes, in the context of a functional derivative of a sequence whether an nucleic acid or amino acid sequence, a molecule that retains a biological activity (either function or structural) that is substantially similar to that of the original sequence. This functional derivative or equivalent may be a natural derivatives or may be prepared synthetically. Such derivatives include amino acid sequences having substitutions, deletions, or additions of one or more amino acids, provided that the biological activity of the protein is conserved. The same applies to derivatives of nucleic acid sequences which can have substitutions, deletions, or additions of one or more nucleotides, provided that the biological activity of the sequence is generally maintained. When relating to a protein sequence, the substituting amino acid as chemico-physical properties which are similar to that of the substituted amino acid. The similar chemico-physical properties include, similarities in charge, bulkiness, hydrophobicity, hydrophylicity and the like. The term “functional derivatives” is intended to include “fragments”, “segments”, “variants”, “analogs” or “chemical derivatives” of the subject matter of the present invention.

Thus, the term “variant” refers herein to a protein or nucleic acid molecule which is substantially similar in structure and biological activity to the protein or nucleic acid of the present invention.

The functional derivatives of the present invention can be synthesized chemically or produced through recombinant DNA technology. all these methods are well known in the art. In view of the conservation of the Ste4p/G_{β} binding domain of Ste20p/PAK throughout evolution (see below), it will be apparent to the person of ordinary skill that sequences from different organisms and animals and chimeras thereof can be used in accordance with the teachings of the present invention.

As used herein, “chemical derivatives” is meant to cover additional chemical moieties not normally part of the subject matter of the invention. Such moieties could affect the physico-chemical characteristic of the derivative (i.e. solubility, absorption, half life and the like, decrease of toxicity). Such moieties are exemplified in Remington's Pharmaceutical Sciences (1980). Methods of coupling these chemical-physical moieties to a polypeptide are well known in the art.

The term “allele” defines an alternative form of a gene which occupies a given locus on a chromosome.

As commonly known, a “mutation” is a detectable change in the genetic material which can be transmitted to a daughter cell. As well known, a mutation can be, for example, a detectable change in one or more deoxyribonucleotide. For example, nucleotides can be added, deleted, substituted for, inverted, or transposed to a new position. Spontaneous mutations and experimentally induced mutations exist. The result of a mutations of nucleic acid molecule is a mutant nucleic acid molecule. A mutant polypeptide can be encoded from this mutant nucleic acid molecule.

As used herein, the term “purified” refers to a molecule having been separated from a cellular component. Thus, for example, a “purified protein” has been purified to a level not found in nature. A “substantially pure” molecule is a molecule that is lacking in all other cellular components.

In general, techniques for preparing antibodies (including monoclonal antibodies and hybridomas) and for detecting antigens using antibodies are well known in the art (Campbell, 1984, In “Monoclonal Antibody Technology: Laboratory Techniques in Biochemistry and Molecular Biology”, Elsevier Science Publisher, Amsterdam, The Netherlands) and in Harlow et al., 1988 (supra). The present invention also provides polyclonal, monoclonal antibodies, or humanized versions thereof, chimeric antibodies and the like which inhibit or neutralize their respective interaction domains and/or are specific thereto.

The term “non-human animals” refers to animals having a transgenic interruption or alteration of an endogenous gene encoding an interaction domain of the present invention (knock-out animal) and/or animals having an interruption into the genome in which a transgene (directing the expression of encoding an interaction domain of, or the present invention) has been introduced. Non-limiting examples of such non-human animals include vertebrates such as rodents, non-human primates, amphibians, reptiles and the like. These animals are prepared in accordance with known methods.

The present invention also relates to a kit for identifying compounds which modulate Ste 20p/PAK—Ste4p/G_{β} interaction, comprising a nucleic acid, a protein or a ligand in accordance with the present invention. For example, a compartmentalized kit in accordance with the present invention includes any kit in which reagents are contained in separate containers. Such containers include small glass containers, plastic containers or strips of plastic or paper. Such containers allow the efficient transfer of reagents from one compartment to another compartment such that the samples and reagents are not cross-contaminated and the agents or solutions of each container can be added in a quantitative fashion from one compartment to another. Such containers could include a container which will accept the test compound, a container which contains the interacting domains used in the assay, containers which contain enzymes, containers which contain wash reagents, and containers which contain the reagents used to detect the extent of interaction of the interacting domains.

The present invention is described in further detail in the following non-limiting examples.

### EXAMPLE 1

Yeast Strains and Manipulations

S. cerevisiae strains used herein were W303-1A (MA Ta ade2 leu2 trp1 ura3 his3 can1), YEL206 (W303-1A ste20Δ-3::TRP1) (Wu et al., 1995, J. Biol. Chem., 270:15984-15992), YEL155 (W303-1A ste5Δ::TRP1) and YEL121 (W303-1A ste4Δ::LEU2). Mating assays, analysis of mating projection formation, and growth arrest, induction of FUSI::lacZ and complementation assays of the growth defect of cells deleted for both STE20 and CLA4 were carried out as described (Leberer et al., 1997, supra; Leberer et al., 1993, Genet., 241:241-254).

### EXAMPLE 2

Construction of Plasmids

To construct pBTL110 carrying HA-STE4P/GB under control of the STE4P promoter, a fragment from nucleotides −491 to −1 of STE4P was amplified by PCR and cloned into pRS313 (Sikorski et al., 1989, Genetics, 122:19-27). The BamHI fragment of pL55 (Whiteway et al., 1995, Science, 269:1572-1575) was then subcloned downstream of the STE4P/GB promoter.

To create pBTL38 and pBTL65 carrying STE4P/GB and HA-STE18 under control of the T3 RNA polymerase promoter, STE4P/GB and HA-STE18 were amplified by PCR and ligated into pRS316 and pRS313 (Sikorski et al., 1989, supra), respectively.

To create pBTL79, pBTL80, pBTL81 and pBTL82 carrying the STE4^{D62N}, STE4^{K55E}, STE4^{N157H/S175P} and STE4^{ΔF177} mutants under control of the T7 RNA polymerase promoter, respectively, the GAL1 promoter was excised from pGAL-STE4P/GB-D62N, pGAL-STE4P/GB-K55E, pGAL-STE4P/GB-N157H/S175P and pGAL-STE4P/GB-ΔF177, respectively (Leberer et al., 1992, supra).

To create pDH171 and pDH172 carrying the Ste20p⁴⁹⁵⁻⁸⁷⁷ and Ste20p⁴⁹⁵⁻⁸⁸⁸ fragments under control of the GAL1 promoter, respectively, these fragments were amplified by PCR and subcloned into pRS313GAL (Leberer et al., 1992, supra).

To create pBTL83, pBTL84, pBTL146 and pBTL147 carrying fusions of GST with the Ste20p⁸⁷⁶⁻⁹³⁹, Ste20p⁸⁷⁶⁻⁸⁹², Ste20p⁸¹⁹⁻⁸⁷⁵ and Ste20p⁸¹⁹⁻⁸⁹² fragments, respectively, fragments were amplified by PCR and subcloned into pGEX-4T-1 (Pharmacia).

To create a fusion of GST with full length Ste5p, the STE5 coding region was amplified by PCR and ligated into pGEX-4T-3 (Pharmacia) to yield pVL50.

### EXAMPLE 3

Oligodeoxynucleotide-directed Mutagenesis of STE20

pBTL151 and pBTL150 carrying the STE20 mutants STE20^{CLA4}, in which the sequence encoding amino acids 879 to 887 of STE20 was replaced by the sequence of CLA4 encoding amino acids 832 to 840 (Cvrckova et al., 1995, supra), and STE20^{S879A/S880A/P883A}, respectively, under control of the STE20 promoter, were created by site directed mutagenesis (Kunkel et al., 1987, Methods in Enzymology, 154:367-382). The mutations were confirmed by sequencing. To create pBTL117 and pBTL118 carrying fusions of GST with the fragments from amino acid 819 to 939 of the STE20^{S879A/S880A/P883A} and STE20^{CLA4} mutants, respectively, these fragments were amplified by PCR and subcloned into pGEX-4T-2 (Pharmacia).

### EXAMPLE 4

Immunochemical Procedures

Immunoprecipitation experiments with specific antibodies to the HA-epitope (12CA5 monoclonal and rabbit polyclonal anti-HA antibodies were from Babco, Richmond), Ste20p (Wu et al., 1995, J. Biol. Chem. 270:15984) and Ste5p (Wall et al., 1995, Cell 83:1047-1058) were performed according to standard procedures as described (Whiteway et al., 1995, supra; Leeuw et al., 1995, supra). For the detection of Ste20p fragments, a secondary sheep antibody specific to rabbit immunoglobulin light chains and a tertiary HRP-conjugated donkey antibody to sheep IgG were obtained from The Binding Site, Lim. Immunoprecipitations were confirmed in at least three independent experiments. For quantitation, immunoblots were evaluated by integrating densitometry using an Epson ES 1200-C densitometer and the NIH Image 1.59 software.

### EXAMPLE 5

In vitro G_{β} Binding Assays

Plasmids were linearized downstream of the termination codons of the respective genes. In vitro-transcription was performed by using either T3 or T7 RNA polymerase and m⁷G(5′)ppp(5′)G capped GTP. In vitro-translation of the resulting mRNA was carried out with ³⁵S-abeled methionine using an in vitro-translation kit (Promega).

GST fusion proteins were purified on glutathione-Sepharose beads in 20 mM HEPES buffer pH 7.4, containing 100 mM NaCl, 50 mM NaF, 0.5 M Sorbitol, 2 mM EDTA, 1 mM Na₃VO₄, 0.1% Triton X-100, 1% BSA (wt/v) and a protease inhibitor cocktail, and washed 5 times in phosphate buffered saline (PBS) (140 mM NaCl, 2.7 mM KCl, 10 mM Na₂HPO₄, 1.8 mM KH₂PO₄, pH 7.4) by centrifugation at 1.000 g. Proteins (5-10 μg) were incubated with 5 μl of the reticulocyte lysate containing the in vitro-translated products in 25 μl of PBS for 10 minutes at 30° C.

The beads were then washed three times with PBS, separated by SDS-PAGE and analysed by autoradiography of Western blots. Results were confirmed in at least three independent experiments. Immunodetection and evaluation of immunoblots and radiographs were then performed as described above. Data obtained for ³⁵S-Ste4p were corrected for relative concentrations of the respective GST fusion proteins.

### EXAMPLE 6

In vivo Association of Ste20p with G_{β} (Ste4p)

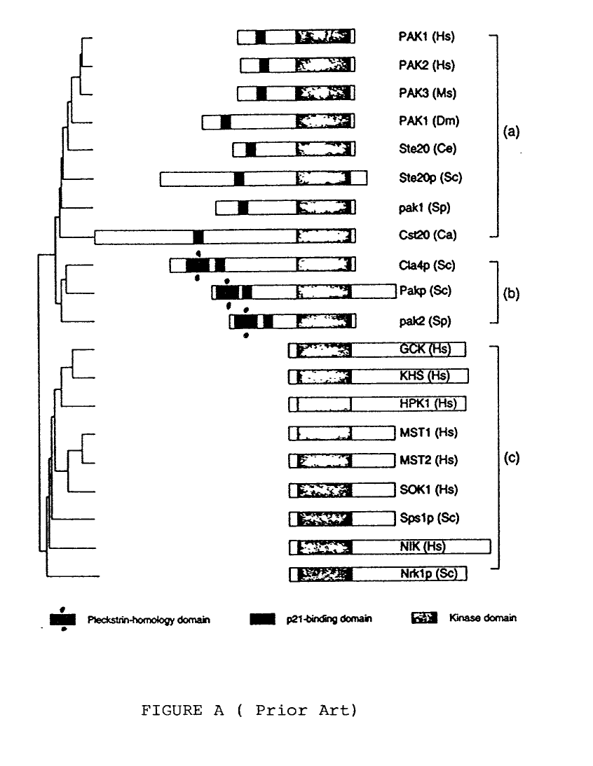

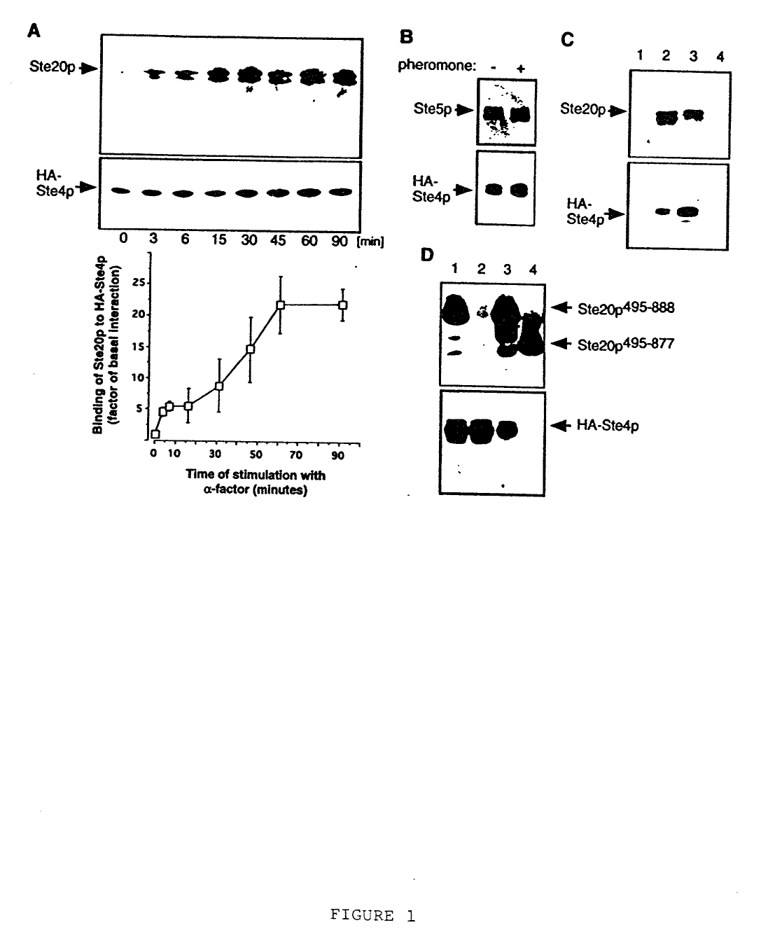

Coimmunoprecipitation experiments were performed to analyze the in vivo-association of Ste20p with an influenza hemagglutinin (HA)-epitope tagged version of G_{β} (HA-Ste4p). Antibodies to HA-Ste4p precipitated low amounts of Ste20p (FIG. 1A). An approximately 5-fold increase in the interaction between Ste4p and Ste20p was observed already after 3 minutes of pheromone treatment and maintained for up to 15 minutes of stimulation (FIG. 1A). The initial induction of Ste20p/Ste4p complexes is consistent with the time course described for the stimulation of Far1p (a cyclin inhibitor) and the MAP kinases Fus3p and Kss1p (Chang et al., 1992, Mol. Biol. Cell., 3:445-450; Gartner et al., 1992, Genes Dev., 6:1280-1292) and may be required to activate the MAP kinase cascade for the induction of growth arrest and transcriptional activation. Additional formation of complexes after prolonged treatment with pheromone (FIG. 1A) followed a time course concomitant with the formation of mating projections and accumulation of receptors, Ste20p and Ste4p (Leberer et al., 1997, Curr. Op. Genet. & Dev., 7:59-66; Leberer et al., 1997, EMBO J., 16:83-97) in the tips of mating projections, and could be involved in the control of morphological changes that may require Ste20p dependent phosphorylation of myosin-l or activation of the PKC pathway (Leberer et al., 1997, EMBO J., 16:83-97; Wu et al., 1996, J. Biol. Chem., 271:31787-31790).

When the pheromone response pathway was activated through overexpression of HA-Ste4p, Ste5p also formed a complex with Ste4p (Whiteway et al., 1995, supra). This complex was present in cells without an activated pathway when HA-Ste4p was expressed at wild-type levels (FIG. 1B), and the association was not significantly altered after treatment of cells with pheromone (FIG. 1B), suggesting a constitutive interaction between Ste4p and Ste5p. Constitutive activation of the pheromone signaling pathway through overexpression of HA-Ste4p stimulated the association of Ste4p with Ste20p in the absence of pheromone (FIG. 1C). This association required the function of Ste18p, the γ-subunit of the mating response G-protein, but did not require the presence of Ste5p (data not shown).

### EXAMPLE 7

Identification of the G_{β} Interaction Domain of Ste20p

Cells overexpressing the Ste20p⁴⁹⁵⁻⁸⁸⁸ fragment were normal in their mating functions, whereas cells overexpressing the Ste20p⁴⁹⁵⁻⁸⁷⁷ fragment were defective (Table 1). Briefly, Strain YEL206 deleted for STE20 was transformed with pDH 166 (Whiteway et al., 1995, supra), pDH171 and pDH172 carrying either wild-type STE20 (STE20^{WT}) or the STE20⁴⁹⁵⁻⁸⁷⁷ and STE20⁴⁹⁵⁻⁸⁸⁸ mutant alleles, respectively, under control of the GAL1 promoter. Mating efficiencies represent mean values ±SD (n=3). Mating functions were analyzed as described (Leberer et al., 1997, supra; Leberer et al., 1993, supra).

TABLE 1
Effects of carboxy-terminal truncations
on signaling functions of Ste20p

| | Mating | FUS1:: lacZ | | | |
|---|---|---|---|---|---|
| STE20 | efficiencies | expression | | | Shmoo |
| allele | (%) | Basal | Induced | G₁ arrest | formation |
| STE20^{WT} | 89.8 ± 15.5 | 0.1 | 221.2 | + | + |
| STE20⁴⁹⁵⁻⁸⁸⁸ | 75.2 ± 7.5 | 1.6 | 147.7 | + | + |
| STE20⁴⁹⁵⁻⁸⁷⁷ | 0.02 ± 0.015 | <0.1 | <0.1 | − | − |

These results suggest that the region from amino acids 877 to 888 carboxyl-terminal to the kinase domain of Ste20p plays an important role in the pheromone response. This region was also required for coimmunoprecipitation with HA-Ste4p (FIG. 1D), underlining the physiological importance of the association between Ste4p and Ste20p in pheromone signaling.

### EXAMPLE 8

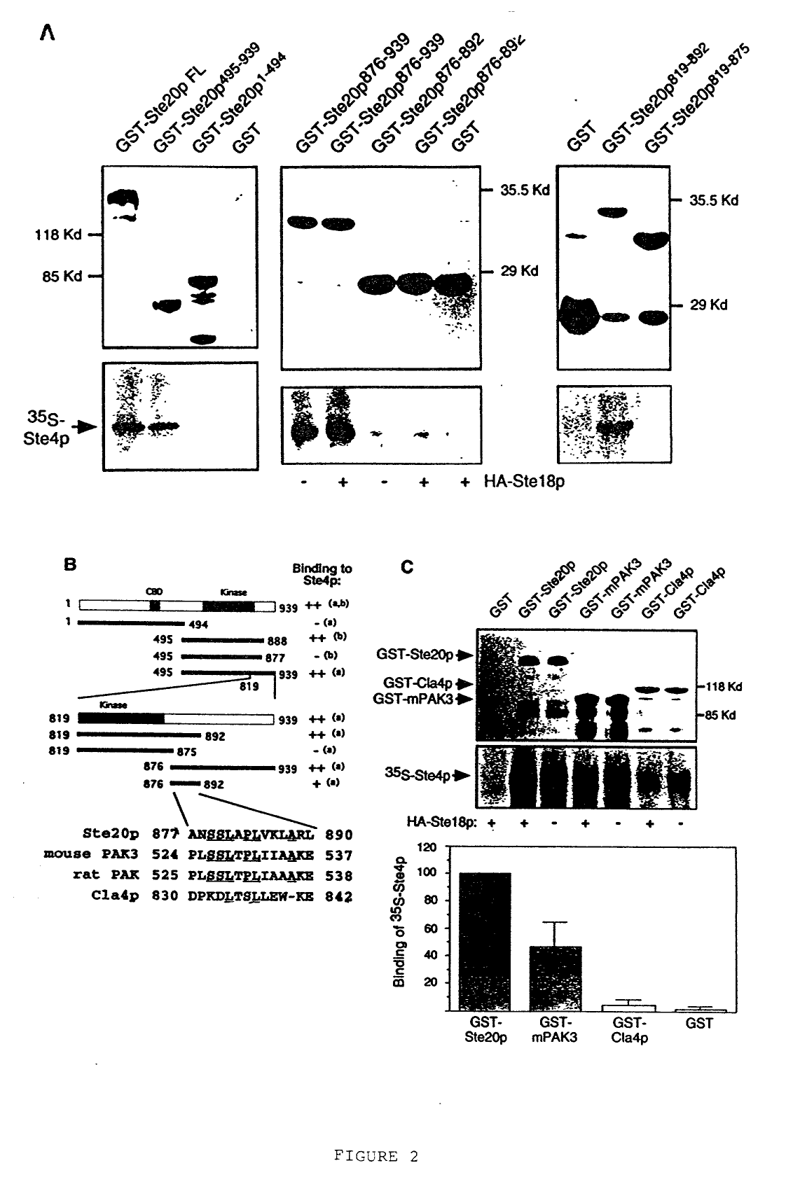

In vitro Association of Ste20p with G_{β} (Ste4p)

[³⁵S]methonine-labeled Ste4p³⁵ (S-Ste4p) and HA-Ste18p were synthesized in an in vitro-translation system and analyzed for their ability to bind to fusions of glutathione S-transferase (GST) with wild-type Ste20p and fragments of Ste20p (FIG. 2A). It was found that a fragment carboxyl-terminal to the kinase domain encompassing residues 876 to 892 was necessary and sufficient to bind ³⁵S-Ste4p (FIG. 2A). The binding did not depend on the presence of HA-Ste18p (FIGS. 2A,C), and HA-Ste18p alone was not able to bind Ste20p (data not shown). As summarized in FIG. 2B, these results, together with data obtained in the immunoprecipitation experiments, suggest that the non-catalytic region from amino acids 876 to 888 of Ste20p represents a binding site for G_{β}.

### EXAMPLE 9

The G_{β} Interaction Domain of Ste20p is Functionally Conserved in Mouse mPAK3 and in Ste20p/PAK Members

Consistent with observations that mammalian PAK isoforms can complement the mating defect of yeast cells deleted for STE20 (Bagrodia et al., 1995, J. Biol. Chem., 270:22731-22737) and that the G_{β}binding site is conserved in these kinases (FIG. 2B), mouse mPAK3 (Bagrodia et al., 1995, supra) bound ³⁵S-Ste4p (FIG. 2C).

### EXAMPLE 10

Identification of the Molecular Determinants of the G_{β} Interaction Domain of Ste20p/PAK—

Ste20p and its closely related isoform Cla4p share a redundant function that is essential for cellular viability in yeast (Cvrckova et al., 1995, supra). Consistent with observations that only high levels of Cla4p after overexpression partly complement the mating defect of yeast cells deleted for STE20 (data not shown) and the Ste4p binding site of Ste20p is not well conserved in Cla4p (FIG. 2B), only weak binding of Ste4p to Cla4p was observed (FIG. 2C). These results support the view that residues conserved in the Ste4p binding sites of Ste20p and PAK isoforms (FIG. 2B) contribute to the binding of G_{β}. These results also provide a weak consensus sequence for G_{β} binding, the sequence of the G_{β} binding domain of Cla4p. This weak G_{β} binding consensus sequence can be used in assays to identify compounds which can stimulate Cla4p-G_{β} interaction. In a particular embodiment, the assay involves the use of agents to identify agonists of the Cla4p-G_{β} interaction that will enable a complementation of the mating defect of yeast cells deleted for Ste20p. In another embodiment, the physical interaction between Cla4p and G_{β} could be assessed in vitro through gel shifts, immunoprecipitation and the like, as well known to the person of ordinary skill and as shown herein. Further, single mutations (or a combination of mutations) in the G-binding domain of Cla4p could identify the minimal primary structure requirements enabling adequate G_{β}-Cla4p binding and perhaps the complementation of the Ste20p null mutant strain mentioned above. A very similar approach is exemplified with single mutations and a triple mutations of the conserved residues of Ste20p (see below).

### EXAMPLE 11

In vitro Mutagenesis of the G_{β} Interaction Domain of a Ste20p/PAK Member

Single alterations of the conserved residues S879, S880 or P883 to alanine did not affect the in vivo-function of Ste20p (data not shown). However, the triple mutant Ste₂₀p^{S879A/S880A/P883A in which the highly conserved sequence motif SSLxPL was altered to AALxAL, showed strong defects in mating functions (Table} 2). Briefly, for GST (control) and fusions of GST with the carboxyl-terminal fragments from amino acids 819 to 939 of wild-type Ste20p (STE20^{WT}), the Ste₂₀p^{S879A/S880A/P883A} mutant and the Ste20^{CLA4} mutant (in which the sequence encoding amino acids 879 to 887 of STE20 was replaced by the sequence of CLA4 encoding amino acids 832 to 840; Cvrckova et al., 1995, supra), respectively, were incubated with in vitro-translated ³⁵S-Ste4p in the presence of in vitro-translated HA-Ste18p. Data are given as relative levels of bound ³⁵S-Ste4p normalized against binding to wild-type Ste20p⁸¹⁹⁻⁹³⁹. For cells deleted for STE20 were transformed with pRS313 (control), pSTE20-5 carrying wild-type STE20 (Leberer et al., 1992, supra) (STE20^{WT}), pBTL150 carrying the STE20^{S879A/S880A/P883A} mutant, and pBTL151 carrying the STE20^{CLA4} mutant. Proteins were expressed under control of the STE20 promoter. Finally in vitro-kinase activities were determined in immune complexes isolated from YEL206 cells expressing the indicated STE20 alleles or the inactive STE20^{K649R} mutant (Wu et al., 1995, supra) as a control. In vitro-kinase assays were performed as described (Wu et al., 1995, supra) with myelin basic protein (MBP) as substrate. Data are given as percentage of MBP phosphorylation by wild-type Ste20p. Similar defects were also observed for the mutant Ste20p^{CLA4}, in which the Ste4p binding site of Ste20p was replaced by the equivalent region of Cla4p (Table 2 and FIG. 2B). No differences were found for the in vitro-kinase activities of these mutants when compared with wild-type Ste20p (Table 2), and the mutants were found to complement the growth defect of cells deleted for both STE20 and CLA4 (data not shown). However, binding to ³⁵S-Ste4p was strongly reduced when fusions of GST with both mutant versions were analyzed in the in vitro-binding assay (Table 2). Thus, the mating defects of these Ste20p mutants correlated with their reduced ability to bind Ste4p.

TABLE 2
Effect of carboxy-terminal mutations in Ste20p
on in vitro-binding to Ste4p, in vivo-signaling
functions and in vitro-kinase activities

| | Binding to | Mating | | | | Kinase |
|---|---|---|---|---|---|---|
| | ³⁵S-Ste4p | efficiencies | FUS1:: lacZ | G₁ | Shmoo | activity |
| STE20 allele | (%)^{a)} | (%)^{b)} | expression^{b)} | arrest^{b)} | formation^{b)} | (%)^{c)} |
| STE20^{WT} | 100 | 72.7 ± 6.8 | 274 ± 41 | + | + | 100 |
| STE20^{S879A/S880A/P883A} | 15.2 ± 7.8 | 0.039 ± 0.002 | 14 ± 1.8 | − | − | 104 ± 11.2 |
| STE20^{CLA4} | 11.1 ± 9.3 | 0.016 ± 0.001 | 11 ± 1.4 | − | − | 93 ± 10.7 |
| Control | 2.1 ± 1.8 | <0.005 | <0.2 ± 1 | − | − | 2.5 ± 1.3 |

Mean values ± SD (n = 3)
^{a, b, c)}see text for details

### EXAMPLE 12

Identification of the Ste20p Binding Domain of Ste4p

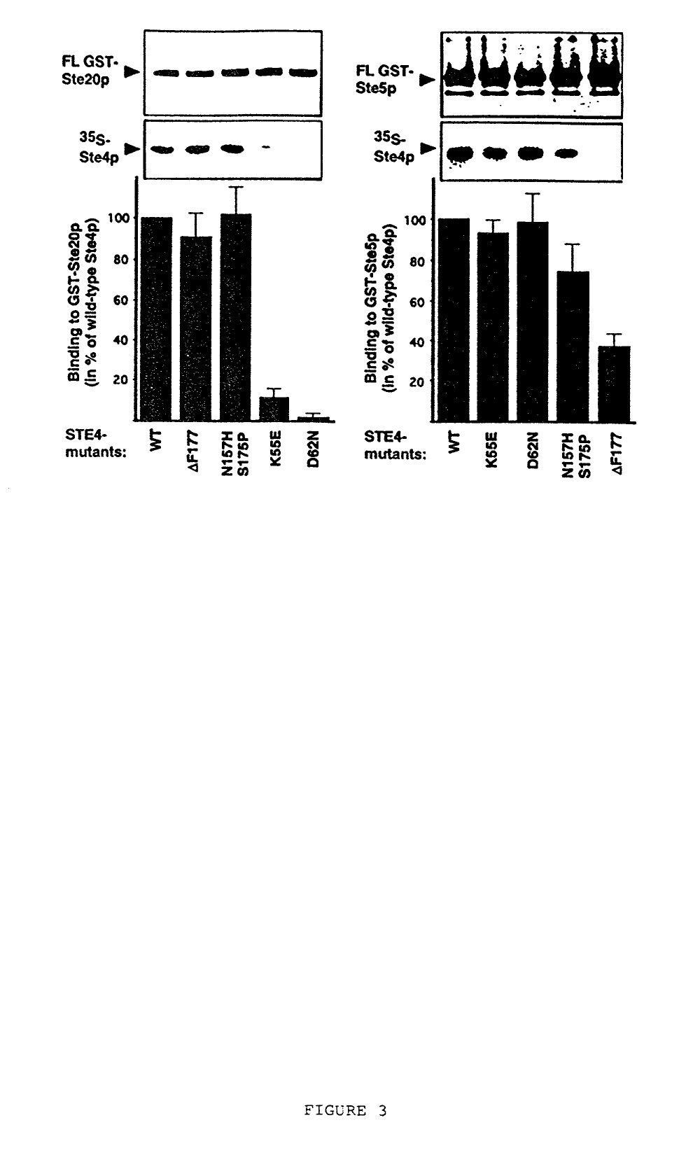

Mutations within two regions of Ste4p which, when overexpressed, inhibited the signaling function of the wild-type protein were previously identified (Leberer et al., 1992, supra). The effect of two of these dominant-negative mutations within each region were examined for their effect on the association of Ste4p with either Ste20p or Ste5p. The K55E and D62N mutants of Ste4p (Leberer et al., 1992, supra) were defective in binding to GST-Ste20p, whereas binding to GST-Ste5p was normal (FIG. 3). The inability of these Ste4p mutants to bind Ste20p correlated with their sterile phenotype (Leberer et al., 1992, supra). However, the N157H/S175P and ΔF177 mutants which were also found to possess reduced signaling functions (Leberer et al., 1992, supra) were able to bind both Ste20p and Ste5p, although binding of Ste5p was reduced when compared with binding to wild-type Ste4p (FIG. 3), suggesting that this region may be involved in the interaction with an as yet unidentified component. The present invention therefore further provides means to identify this unidentified component and a further dissection of the structure-function relationship of Ste20p/PAK in signalling function.

Modeling of Ste4p by using the crystal structure of mammalian G_{β1} (Wall et al., 1995, Cell 83:1047-1058) as a template indicates that the residues predicted to interact with Ste20p are part of an amino-terminal a-helix in the region of G_{β} that interacts with G_{γ} (Wall et al., 1995, supra; Sondek et al., 1996, Nature, 379:369-374). The structure of yeast Ste4p (G_{β}) was modelled to the structure of mammalian G_{β1} (Wall et al., supra) using the homology module of Insight (Biosym, Inc.). Insertions specific for Ste4p were not considered.

However, consistent with the finding that the Ste4p^{D62N} mutant interacted normally with Ste18p in the two-hybrid system (data not shown), the side chains of these residues are not predicted to be involved in the interaction with G_{γ} but rather to be exposed on the cytoplasmic face of the G_{β} structure (data not shown).

Conclusion

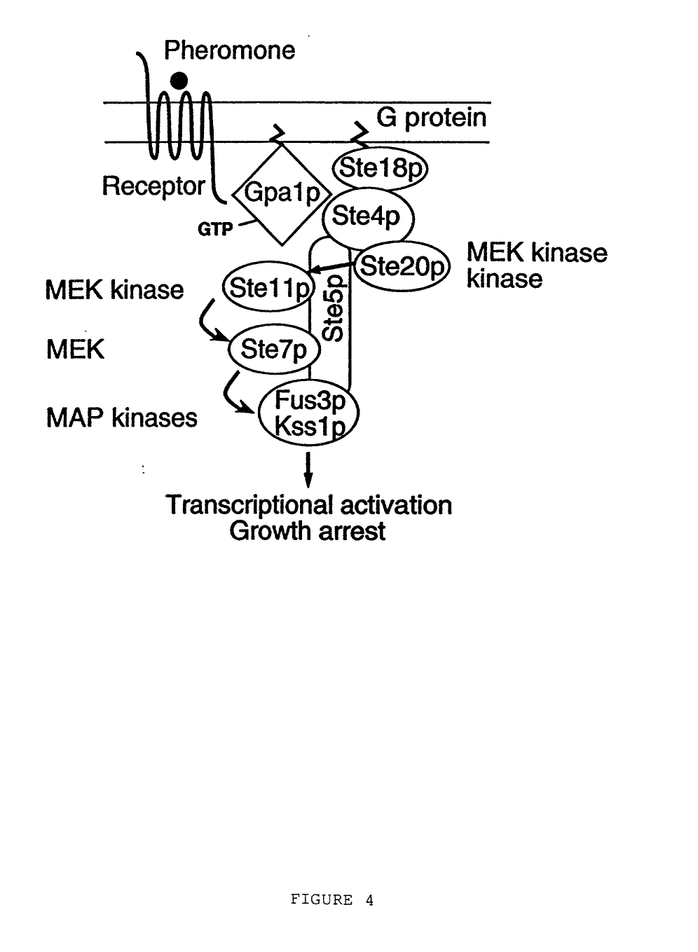

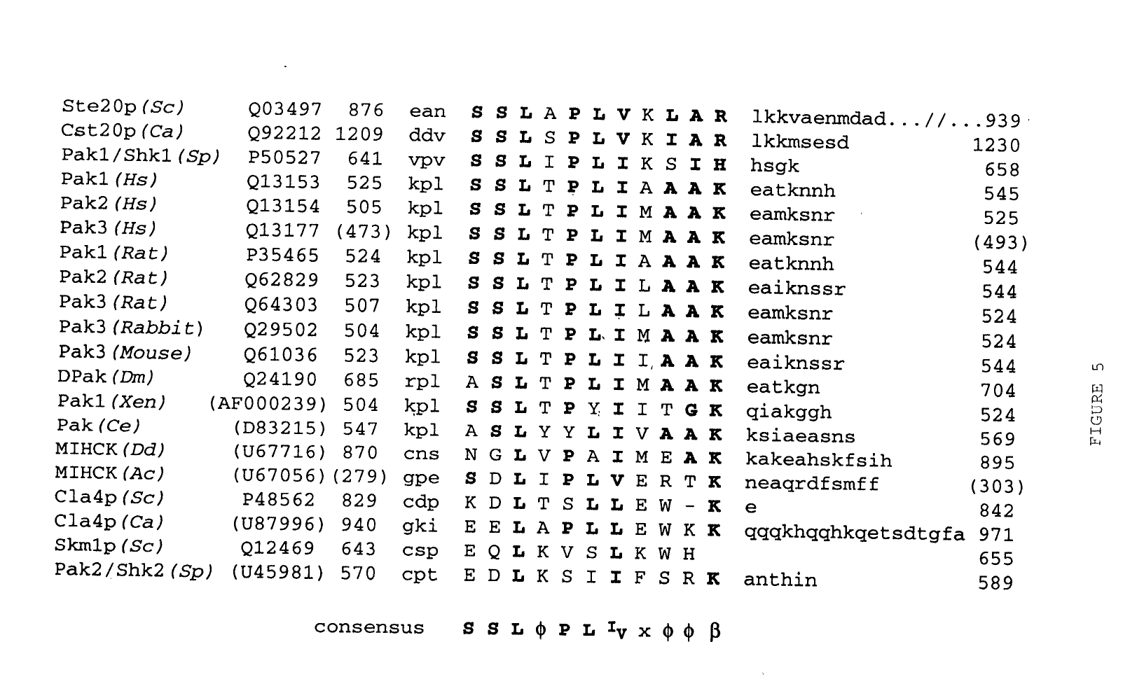

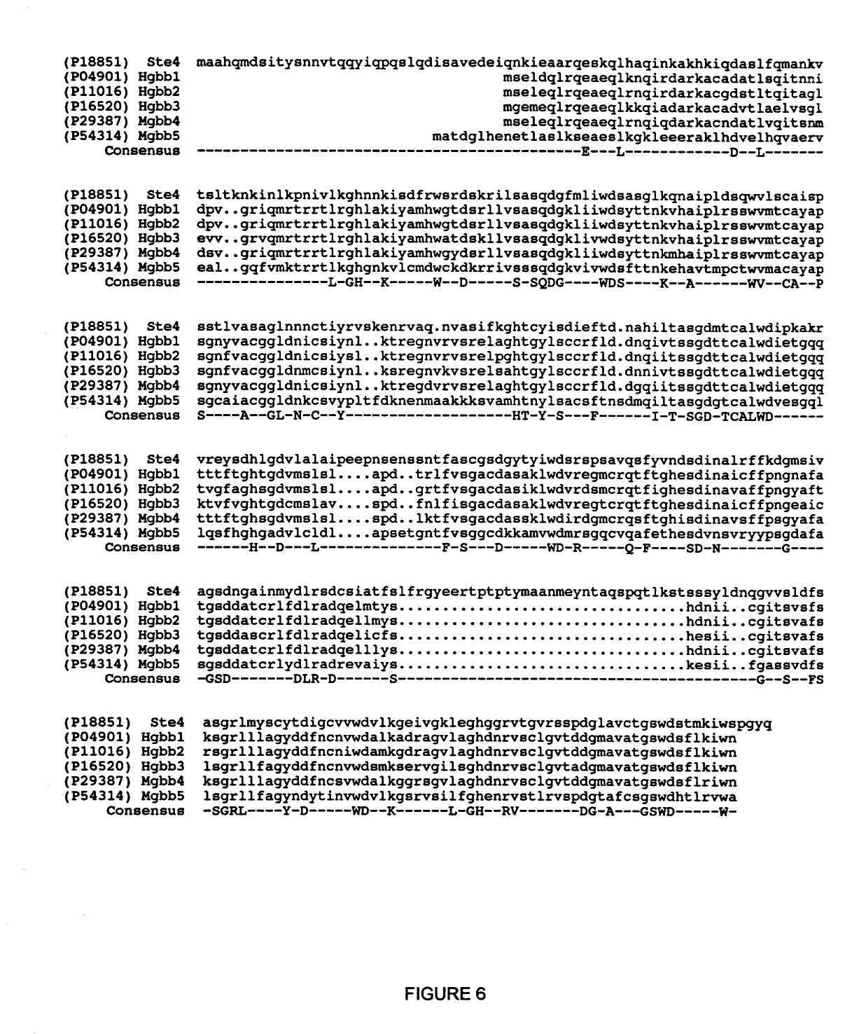

Together, these results indicate that transmission of the pheromone signal involves the regulated interaction between the mating-response G-protein β-subunit and a conserved sequence in the Ste20p protein kinase (FIG. 4). Pheromone-induced interaction with Ste4p may bring Ste20p in vicinity of Ste11p (FIG. 4) which interacts with Ste5p (Leberer et al., 1997, Curr. Op. Genet. & Dev., 7:59-66) and can serve as an in vitro-substrate for Ste20p (Wu et al., 1995, J. Biol. Chem., supra). Low concentrations of Ste20p/G_{β} complexes present in the absence of pheromone may account for the basal signalling levels found in uninduced cells and may guarantee the rapid responsiveness of cells to pheromone (Chang et al., 1992, supra; Gartner et al., 1992, supra). In view of the high degree of conservation of Ste20p family protein kinases (Sells et al, 1997, supra), the results presented herein suggest that the interaction of these kinases with the β-subunit of heterotrimeric G-proteins (which are also highly conserved) may contribute to linking Ste20p homologs to G-protein-coupled receptors in other organisms including mammalian cells.

Although the present invention has been described hereinabove by way of preferred embodiments thereof, it can be modified, without departing from the spirit and nature of the subject invention as defined in the appended claims.

What is claimed is:

1. An isolated Ste4p/G_{β}-binding polypeptide or fragment thereof wherein said isolated Ste4p/G_{β}-binding polypeptide is a Ste20p/PAK polypeptide which directly binds to a Ste4p/G_{β} polypeptide or fragment thereof.

2. The isolated Ste4p/G_{β}-binding polypeptide of claim 1, comprising an amino acid sequence having at least 95% identity to the amino acid sequence selected from the group consisting of:
a) a full length amino acid sequence of SEQ. ID. NO.:29;
b) an amino acid sequence having amino acids 495 to 939 of SEQ. ID. NO.:29;
c) an amino acid sequence having amino acids 495 to 888 of SEQ. ID. NO.:29;
d) an amino acid sequence having amino acids 819 to 939 of SEQ. ID. NO.:29;
e) an amino acid sequence having amino acids 819 to 892 of SEQ. ID. NO.:29;
f) an amino acid sequence having amino acids 876 to 939 of SEQ. ID. NO.:29; and
g) an amino acid sequence having amino acids 876 to 892 of SEQ. ID. NO.:29.

3. The isolated Ste4p/G_{β}-binding polypeptide of claim 1, comprising an amino acid sequence having at least 95% identity to the amino acid sequence selected from the group consisting of:
a) an amino acid sequence of SEQ. ID. NO.:29;
b) an amino acid sequence of SEQ. ID. NO.: 1;
c) an amino acid sequence of SEQ. ID. NO.:2;
d) an amino acid sequence of SEQ. ID. NO.:3;
e) an amino acid sequence of SEQ. ID. NO.:4;
e) an amino acid sequence of SEQ. ID. NO.:5;
g) an amino acid sequence of SEQ. ID. NO.:6;
h) an amino acid sequence of SEQ. ID. NO.:7;
i) an amino acid sequence of SEQ. ID. NO.:8;
j) an amino acid sequence of SEQ. ID. NO.:9;
k) an amino acid sequence of SEQ. ID. NO.:10
l) an amino acid sequence of SEQ. ID. NO.:11;
m) an amino acid sequence of SEQ. ID. NO.:12; and
n) an amino acid sequence of SEQ. ID. NO.:13.

4. The isolated Ste4p/G_{β}binding polypeptide of claim 1, joined to a heterologous polypeptide, thereby forming an isolated chimeric polypeptide which directly binds to a Ste4p/G_{β} polypeptide or fragment thereof.

5. The isolated Ste4p/G_{β}-binding polypeptide of claim 4, comprising amino acid sequence SSLφPLI_{V}Xφφβ, wherein φ is selected from A, I, L, M, S, T, and β is selected from H, K, and R.

6. The isolated Ste4p/G_{β}-binding polypeptide of claim 5, wherein said heterologous polypeptide is Glutathione-S-transferase.

7. An isolated nucleic acid molecule comprising a nucleic acid sequence which encodes a Ste4p/G_{β} binding domain of Ste20p/PAK.

8. The isolated nucleic acid of claim 7, comprising a nucleic acid sequence at least 90% identical to a sequence selected from the group consisting of:
a) a nucleotide sequence encoding a full length amino acid sequence of SEQ. ID. NO.:29;
b) a nucleotide sequence encoding an amino acid sequence having amino acid 495 to 939 of SEQ. ID. NO.:29;
c) a nucleotide sequence encoding an amino acid sequence having amino acid 495 to 888 of SEQ. ID. NO.:29;
d) a nucleotide sequence encoding an amino acid sequence having amino acid 819 to 939 of SEQ. ID. NO.:29;
e) a nucleotide sequence encoding an amino acid sequence having amino acid 819 to 892 of SEQ. ID. NO.:29;
f) a nucleotide sequence encoding an amino acid sequence having amino acid 876 to 939 of SEQ. ID. NO.:29;
g) a nucleotide sequence encoding an amino acid sequence having amino acid 876 to 892 of SEQ. ID. NO.:29; and
h) a nucleotide sequence which hybridizes to a)-g) under high stringency conditions.

9. The isolated nucleic acid of claim 7, comprising a nucleic acid sequence at least 90% identical to a sequence selected from the group consisting of:
a) a nucleotide sequence encoding an amino acid sequence of SEQ. ID. NO.:29;
b) a nucleotide sequence encoding an amino acid sequence of SEQ. ID. NO.:1;
c) a nucleotide sequence encoding an amino acid sequence of SEQ. ID. NO.:2;
d) a nucleotide sequence encoding an amino acid sequence of SEQ. ID. NO.:3;
e) a nucleotide sequence encoding an amino acid sequence of SEQ. ID. NO.:4;
f) a nucleotide sequence encoding an amino acid sequence of SEQ. ID. NO.:5;
g) a nucleotide sequence encoding an amino acid sequence of SEQ. ID. NO.:6;
h) a nucleotide sequence encoding an amino acid sequence of SEQ. ID. NO.:7;
i) a nucleotide sequence encoding an amino acid sequence of SEQ. ID. NO.:8;
j) a nucleotide sequence encoding an amino acid sequence of SEQ. ID. NO.:9;
k) a nucleotide sequence encoding an amino acid sequence of SEQ. ID. NO.:10;
l) a nucleotide sequence encoding an amino acid sequence of SEQ. ID. NO.: 11;
m) a nucleotide sequence encoding an amino acid sequence of SEQ. ID. NO.:12; and
n) a nucleotide sequence encoding an amino acid sequence of SEQ. ID. NO.:13;
o) a nucleotide sequence encoding an amino acid sequence SSLφPLI_{V}Xφφβ, wherein φ is selected from A, I, L, M, S, T, and β is selected from H, K, and R;
p) a nucleotide sequence which hybridizes to a)-e) under high stringency conditions.

10. An isolated nucleic acid molecule encoding a Ste4p/G_{β} binding domain containing a fusion protein, said Ste4p/G_{β} interaction domain being fused to a heterologous polypeptide sequence, wherein said Ste4p/G_{β} binding domain is encoded by the nucleic acid molecule of claim 7.

11. The isolated nucleic acid molecule of claim 10 wherein said Ste4p/G_{β} binding domain comprises amino acid sequence SSLφPLI_{V}Xφφβ, wherein φ is selected from A, I, L, M, S, T, and β is selected from H, K, and R.

12. The isolated nucleic acid molecule of claim 11, wherein said heterologous polypeptide sequence encodes Glutathione-S-transferase.

13. An isolated nucleic acid molecule comprising a nucleic acid sequence which encodes a Ste20p/PAK binding domain of Ste4p/G_{β}, wherein said Ste 20p/PAK binding domain is a Ste4p/G_{β} polypeptide which directly binds to a Ste20p/PAK polypeptide or fragment thereof.

14. The isolated nucleic acid molecule of claim 13, wherein said sequence encodes a polypeptide comprising an amino acid sequence having at least 95% identity to the amino acid sequence selected from the group consisting of:
a) a full length amino acid sequence of SEQ. ID. NO.:21;
b) an amino acid sequence having amino acids 1 to 150 of SEQ. ID. NO.:21;
c) an amino acid sequence having amino acids 1 to 100 of SEQ. ID. NO.:21;
d) an amino acid sequence having amino acids 1 to 80 of SEQ. ID. NO.:21;
e) an amino acid sequence of SEQ. ID. NO.:22;
f) an amino acid sequence of SEQ. ID. NO.:23;
g) an amino acid sequence of SEQ. ID. NO.:24;
h) an amino acid sequence of SEQ. ID. NO.:25; and
i) an amino acid sequence of SEQ. ID. NO.:26.

15. A vector comprising the nucleic acid molecule of claim 7.

16. A host cell harboring the nucleic acid molecule of claim 15.

17. The host cell of claim 16 further harboring a vector comprising a nucleic acid molecule which comprises a nucleic acid sequence encoding a Ste20p/PAK interaction domain of Ste4p/G_{β}.

18. The host cell of claim 17, wherein at least one assayable metabolic function is dependent on the interaction of said nucleic acid sequences encoding the Ste4p/G_{β} interaction domain of Ste20p/PAK and the Ste20p/PAK interaction domain of Ste4p/G_{β}.

19. A method of assaying compounds having the ability to modulate the interaction between Ste20p/PAK and Ste4p/G_{β} comprising the steps of:
a) incubating the host cell of claim 18 with a test compound;
b) assaying said at least one metabolic function dependent on said interaction between Ste20p/PAK and Ste4p/G_{β}; and
c) identifying said compound as a modulator of said interaction.

20. A method of assaying compounds having the ability to modulate the interaction between Ste20p/PAK and Ste4p/G_{β} comprising the steps of:
a) incubating the isolated Ste4p/G_{β} polypeptide of claim 5 with an isolated Ste20p/PAK-binding polypeptide or fragment thereof, wherein said isolated Ste20p/PAK-binding polypeptide is a Ste4p/G_{β} polypeptide which directly binds to a Ste20p/PAK polypeptide or fragment thereof, in the presence of a test compound;
b) assaying in vitro the binding between said isolated Ste4p/G_{β} polypeptide and said isolated Ste20p/PAK polypeptide; and
c) identifying said compound as a modulator of said interaction.


SEQUENCE LISTING (31 sequences)

SEQ ID NO: 1 - protein, 423 aa, Unknown Organism
  Description of Unknown Organism unknown
MAAHQMDSITYSNNVTQQYIQPQSLQDISAVEDEIQNKIEAARQESKQLHAQINKAKHKIQDASLFQMANKVTSLTKNKINLKPNIVLKGHNNKISDFRWSRDSKRILSASQDGFMLIWDSASGLKQNAIPLDSQWVLSCAISPSSTLVASAGLNNNCTIYRVSKENRVAQNVASIFKGHTCYISDIEFTDNAHILTASGDMTCALWDIPKAKRVREYSDHLGDVLALAIPEEPNSENSSNTFASCGSDGYTYIWDSRSPSAVQSFYVNDSDINALRFFKDGMSIVAGSDNGAINMYDLRSDCSIATFSLFRGYEERTPTPTYMAANMEYNTAQSPQTLKSTSSSYLDNQGVVSLDFSASGRLMYSCYTDIGCVVWDVLKGEIVGKLEGHGGRVTGVRSSPDGLAVCTGSWDSTMKIWSPGYQ

SEQ ID NO: 2 - protein, 340 aa, Unknown Organism [sequence omitted]

SEQ ID NO: 3 - protein, 340 aa, Unknown Organism
  Description of Unknown Organism unknown
MSELEQLRQEAEQLRNQIRDARKACGDSTLTQITAGLDPVGRIQMRTRRTLRGHLAKIYAMHWGTDSRLLVSASQDGKLIIWDSYTTNKVHAIPLRSSWVMTCAYAPSGNFVACGGLDNICSIYSLKTREGNVRVSRELPGHTGYLSCCRFLDDNQIITSSGDTTCALWDIETGQQTVGFAGHSGDVMSLSLAPDGRTFVSGACDASIKLWDVRDSMCRQTFIGHESDINAVAFFPNGYAFTTGSDDATCRLFDLRADQELLMYSHDNIICGITSVAFSRSGRLLLAGYDDFNCNIWDAMKGDRAGVLAGHDNRVSCLGVTDDGMAVATGSWDSFLKIWN

SEQ ID NO: 4 - protein, 340 aa, Unknown Organism [sequence omitted]

SEQ ID NO: 5 - protein, 340 aa, Unknown Organism
  Description of Unknown Organism unknown
MSELEQLRQEAEQLRNQIQDARKACNDATLVQITSNMDSVGRIQMRTRRTLRGHLAKIYAMHWGYDSRLLVSASQDGKLIIWDSYTTNKMHAIPLRSSWVMTCAYAPSGNYVACGGLDNICSIYNLKTREGDVRVSRELAGHTGYLSCCRFLDDGQIITSSGDTTCALWDIETGQQTTTFTGHSGDVMSLSLSPDLKTFVSGACDASSKLWDIRDGMCRQSFTGHISDINAVSFFPSGYAFATGSDDATCRLFDLRADQELLLYSHDNIICGITSVAFSKSGRLLLAGYDDFNCSVWDALKGGRSGVLAGHDNRVSCLGVTDDGMAVATGSWDSFLRIWN

SEQ ID NO: 6 - protein, 353 aa, Unknown Organism
  Description of Unknown Organism unknown
MATDGLHENETLASLKSEAESLKGKLEEERAKLHDVELHQVAERVEALGQFVMKTRRTLKGHGNKVLCMDWCKDKRRIVSSSQDGKVIVWDSFTTNKEHAVTMPCTWVMACAYAPSGCAIACGGLDNKCSVYPLTFDKNENMAAKKKSVAMHTNYLSACSFTNSDMQILTASGDGTCALWDVESGQLLQSFHGHGADVLCLDLAPSETGNTFVSGGCDKKAMVWDMRSGQCVQAFETHESDVNSVRYYPSGDAFASGSDDATCRLYDLRADREVAIYSKESIIFGASSVDFSLSGRLLFAGYNDYTINVWDVLKGSRVSILFGHENRVSTLRVSPDGTAFCSGSWDHTLRVWA

SEQ ID NO: 7 - protein, 25 aa, Unknown Organism
  Description of Unknown Organism unknown
EANSSLAPLVKLARLKKVAENMDAD

SEQ ID NO: 8 - protein, 22 aa, Unknown Organism
  Description of Unknown Organism unknown
DDVSSLSPLVKIARLKKMSESD

SEQ ID NO: 9 - protein, 18 aa, Unknown Organism
  Description of Unknown Organism unknown
VPVSSLIPLIKSIHHSGK

SEQ ID NO: 10 - protein, 21 aa, Unknown Organism
  Description of Unknown Organism unknown
KPLSSLTPLIAAAKEATKNNH

SEQ ID NO: 11 - protein, 21 aa, Unknown Organism
  Description of Unknown Organism unknown
KPLSSLTPLIMAAKEAMKSNR

SEQ ID NO: 12 - protein, 21 aa, Unknown Organism
  Description of Unknown Organism unknown
KPLSSLTPLIMAAKEAMKSNR

SEQ ID NO: 13 - protein, 21 aa, Unknown Organism
  Description of Unknown Organism unknown
KPLSSLTPLIAAAKEATKNNH

SEQ ID NO: 14 - protein, 22 aa, Unknown Organism
  Description of Unknown Organism unknown
KPLSSLTPLILAAKEAIKNSSR

SEQ ID NO: 15 - protein, 21 aa, Unknown Organism
  Description of Unknown Organism unknown
KPLSSLTPLILAAKEAMKSNR

SEQ ID NO: 16 - protein, 21 aa, Unknown Organism
  Description of Unknown Organism unknown
KPLSSLTPLIMAAKEAMKSNR

SEQ ID NO: 17 - protein, 22 aa, Unknown Organism
  Description of Unknown Organism unknown
KPLSSLTPLIIAAKEAIKNSSR

SEQ ID NO: 18 - protein, 20 aa, Unknown Organism
  Description of Unknown Organism unknown
RPLASLTPLIMAAKEATKGN

SEQ ID NO: 19 - protein, 21 aa, Unknown Organism
  Description of Unknown Organism unknown
KPLSSLTPYIITGKQIAKGGH

SEQ ID NO: 20 - protein, 23 aa, Unknown Organism
  Description of Unknown Organism unknown
KPLASLYYLIVAAKKSIAEASNS

SEQ ID NO: 21 - protein, 26 aa, Unknown Organism
  Description of Unknown Organism unknown
CNCNGLVPAIMEAKKAKEAHSKFSIH

SEQ ID NO: 22 - protein, 25 aa, Unknown Organism
  Description of Unknown Organism unknown
GPESDLIPLVERTKNEAQRDFSMFF

SEQ ID NO: 23 - protein, 14 aa, Unknown Organism
  Description of Unknown Organism unknown
CDPKDLTSLLEWKE

SEQ ID NO: 24 - protein, 32 aa, Unknown Organism
  Description of Unknown Organism unknown
GKIEELAPLLEWKKQQQKHQQHKQETSDTGFA

SEQ ID NO: 25 - protein, 13 aa, Unknown Organism
  Description of Unknown Organism unknown
CSPEQLKVSLKWH

SEQ ID NO: 26 - protein, 20 aa, Unknown Organism
  Description of Unknown Organism unknown
CPTEDLKSIIFSRKANTHIN

SEQ ID NO: 27 - protein, 11 aa, Artificial Sequence
  Description of Artificial Sequence consensus sequence
SSLXPLXXXXX

SEQ ID NO: 28 - DNA, 4136 bp, Saccharomyces cerevisiae
  CDS (276)..(3092)
gaattcgaaagtctaccgcttttggcagctgaaaaattcagaaagtcacctggccagagaggaaaaatacgaaaccaaaaagaggcatccgtaaattcgcattagcaacgcatgcttacatagatactcacatactacacacacttacatactttcttaaagacatacatccgtacgtacaattagagcgaggtagcaagcaacccaaacttcttcccttcactgcctcacaccccatcctaaatatcccacaagatcctcgactaatacaagaaatgagcaatgatccatctgctgtatcggaactaccagacaaggacagtcttgataacggtatcagcaatgacaatgaaagggccatgggcggcaatggcgatggcggcgatggattacgattaccaaggaccactggaactttgaacgtcaatgccttacaaaaaggcactaatgctgcccatgaagctggtggatacaaatccatggatcctgcgaagaacgcggagacaaccaatgatgatgacaataatgtcgtttcactagatgatcctattcaatttacccgagtatcttcctcctctgtcatcagtggaatgtcttcatccatgagtcctcattctaacatcgatgaaaccaaatctctagaagcagtcactccaaacataaataccagcaatataaccccggatcattctgctgacaacacattttctaccataaatgcgtccgagtcagatcaccagtttaatgacactttactatcaaaactgtcgttaacagattctacagaaactatagaaaataacgcgacagtgaagcaccagcagccagttgcatcttccacagtaaactcgaataagagctccactgatatacgaagggctacaccagtgtccactcccgttatctctaaaccatcgatgacaaccacgccaagacagatcaattcagcttcccattcgctttcgaaccctaagcataagcaacataaaccaaaagttaaaccgtccaagcctgaagcaaaaagtaaaccggtttctgtgaaaaaaagctttccttcgaaaaatcctttaaaaaactcctctccacctaaaaagcaaacagaaaaatcgtattattcttcctcttcgaaaaaaaggaaaagcggttcaaatagtggtacactaagaatgaaagatgtctttacgtcctttgtacagaatataaagagaaattctcaggatgataaaagggcctcatcgtcgtccaataattcttcctcatcttctataaccaccgctttgaggatatctacgccatacaatgccaagcatatccaccatgtgggcgtggactccaagactggtgagtacacaggtttgccggaggaatgggaaaaattgttgacttctagtggtatttccaaaagagaacaacagcaaaacatgcaagcagtcatggatattgtcaaattctatcaggatgtcacggaaacaaacggtgaagataaaatgttcaagactttcaacacaaccacaggattgccgggaagtcctcaagtttcaacaccgcctgcaaactcattcaataaatttcctccgtcgacaagtgattcgcacaattacggttccagaacaggtacaccaatgtccaatcacgtcatgtctccaaccttaaatacagattctagttcagcaaacgggaaattcataccaagtagaccggctcctaagcccccatcttctgcgtccgcttcagctccaattataaaatcacccgtcatgaattctgccgccaatgtttcgcccttgaagcagactcatgcacctacaactccgaacaggaccagcccaaacaggtcctcaatatcaagaaatgccactttaaaaaaagaggagcagccactaccaccaatacctccaaccaaatccaaaacgtctccaatcatctccacagctcacacaccacagcaagttgctcaatcgccaaaagcgccggcgcaagagacggtaacgacacctacttcgaagccagctcaagcaagaagcttgtctaaagaattaaatgagaaaaagagagaggaaagggaaagacgtaaaaaacaactatatgccaaattgaacgaaatttgctcagacggtgacccaagtacaaaatatgccaatttagtaaaaattggtcaaggtgcatcaggtggtgtttatactgcttatgaaataggtacgaatgtctcagtggccattaagcaaatgaatctcgaaaagcaaccaaaaaaggagctaatcatcaatgagattctggtcatgaagggtagcaaacaccctaatatagttaatttcattgattcttacgttttaaaaggcgacctttgggtcattatggaatacatggaaggtggctccttaactgatgtggtcacccattgtattttgacagaaggtcaaattggtgccgtttgtagagaaactttgagtgggttggaatttttacattctaaaggtgttcttcacagagatatcaaatccgataacatcctattgtccatggaaggggatattaagttaacggatttcggtttttgcgctcaaatcaatgaattgaacttgaaaagaactactatggtgggaacgccttattggatggcgcctgaagtggtttctaggaaagaatatggcccaaaagtagatatctggtcgttgggtatcatgatcattgaaatgatcgagggggagcctccatatttaaatgaaaccccgctaagagcactgtatttaattgctacaaatggtacacccaagttaaaggaacccgagaatctatcgtcaagcttgaaaaaattccttgattggtgtttatgtgtggagcccgaagatagagcaagcgctacggaattgcttcatgatgaatatatcacggagatagctgaagccaattcctcattggccccgctagtcaagttagcaagattgaagaaagtagctgagaacatggatgctgatgaagataatgacgacgataacgacaacgagcatattaataagacaaacaattgtgacgacaataacgatagcaaagaaaccgtaaatttggacgtaactgaagatgataaacaaaagtaaacgtagcaagcagggtacaccttattatcgacaaagtatatacacagttgtgactggcataaaaattcttttcatatatcttatcgtgtatatttggacattttataacacatcccactctaattcacaacttcattaacgaaatttaaataaatcacgacaacagttttgcttaaaactgaggaatattgaaaccaactcaaattcttcctaatttcaggcgtataaaaataacaaattctcatcgattgtcgggtaccattacacgaacatctgtctgcgttctatgtaacgaaggagaggtattatccaattttggaaatatccgtaatattgtccttagtgcacgaactatattatcccgcaaattcagggaaaagaaaagaagtagaaaaaaaaaaataccatgggagtcagttcttgttcagctgagagaattacgcttgtttcttatttcccacatatacgagaaattcctaccgatataacatcctctctcgtcttctagaattttccagttgagtgaagttttttattttcataaactaacaagattatttcatggaacagtgacggaaaggattttctaaaggcattgttagaaaaaatggttgacgactcaaactatcttacaccacatgaaactgcattagcggtggtggccactgcaatgaagaaagcaagactgcaactagatacattgctaataaattccatacttggtggcgttctgtttagtagtggttcgttctattggtagcggtatattccgaagatcctgacatagtcgcacgaaacccgggtattgtgaatcttattactggtgttaatttcgccatgggactattctatgtagtaatgatgggtgctgacctcttcaactctaatatcctatttttctccgttggagttctgagaaaagcagtaactatctatgatttgatgatttcgtgggttgtcagttggttaggtaatattgctggctcactttttgtttcatatctttttggtcatctttctggtattagttctcagaagctt

SEQ ID NO: 29 - protein, 939 aa, Saccharomyces cerevisiae
MSNDPSAVSELPDKDSLDNGISNDNERAMGGNGDGGDGLRLPRTTGTLNVNALQKGTNAAHEAGGYKSMDPAKNAETTNDDDNNVVSLDDPIQFTRVSSSSVISGMSSSMSPHSNIDETKSLEAVTPNINTSNITPDHSADNTFSTINASESDHQFNDTLLSKLSLTDSTETIENNATVKHQQPVASSTVNSNKSSTDIRRATPVSTPVISKPSMTTTPRQINSASHSLSNPKHKQHKPKVKPSKPEAKSKPVSVKKSFPSKNPLKNSSPPKKQTEKSYYSSSSKKRKSGSNSGTLRMKDVFTSFVQNIKRNSQDDKRASSSSNNSSSSSITTALRISTPYNAKHIHHVGVDSKTGEYTGLPEEWEKLLTSSGISKREQQQNMQAVMDIVKFYQDVTETNGEDKMFKTFNTTTGLPGSPQVSTPPANSFNKFPPSTSDSHNYGSRTGTPMSNHVMSPTLNTDSSSANGKFIPSRPAPKPPSSASASAPIIKSPVMNSAANVSPLKQTHAPTTPNRTSPNRSSISRNATLKKEEQPLPPIPPTKSKTSPIISTAHTPQQVAQSPKAPAQETVTTPTSKPAQARSLSKELNEKKREERERRKKQLYAKLNEICSDGDPSTKYANLVKIGQGASGGVYTAYEIGTNVSVAIKQMNLEKQPKKELIINEILVMKGSKHPNIVNFIDSYVLKGDLWVIMEYMEGGSLTDVVTHCILTEGQIGAVCRETLSGLEFLHSKGVLHRDIKSDNILLSMEGDIKLTDFGFCAQINELNLKRTTMVGTPYWMAPEVVSRKEYGPKVDIWSLGIMIIEMIEGEPPYLNETPLRALYLIATNGTPKLKEPENLSSSLKKFLDWCLCVEPEDRASATELLHDEYITEIAEANSSLAPLVKLARLKKVAENMDADEDNDDDNDNEHINKTNNCDDNNDSKETVNLDVTEDDKQK

SEQ ID NO: 30 - DNA, 2325 bp, Saccharomyces cerevisiae
  CDS (496)..(1764)
gaaaaatgtttcaggaagagatactgcgtaaaaaaaagacacatgtgttacgcaggaaaaagtttgtgaggctttttgccttaacagattgacttgtagccctgttaggtttacccaacatttgtttttctgtgtgtcgaaaattttttcagagtgttttcaactgacacttgcctgtttcatattagttgtaacttaaactttcaaacataaaacttttttggaagtccatccttcacatgacttgaatcccttcaatatcgaaacagttatcctcaaaatctcttatcacttttctaattgttttcttccccttttttgtagtaactcgctgtaaagcacattttattcataatctcctttgtgccagaactcaaggtcaataggccagaattattggaaggaaagagggaagaaaatacgatattgctagttcattaagtcaaggaagaaaatactcaaaaaactgtacagctcaatcaggtacacattacgatggcagcacatcagatggactcgataacgtattctaataatgtcacccaacagtatatacaaccacaaagtctacaggatatctctgcagtggaggaagaaattcaaaataaaatagaggccgccagacaagagagtaaacagcttcatgctcaaataaataaagcaaaacacaagatacaagatgcaagcttattccagatggccaacaaagttacttcgttgaccaaaaataagatcaacttaaagccaaatatcgtgttgaaaggccataataataaaatctcagattttcggtggagtcgagattcaaaacgtattttgagtgcaagtcaagatggctttatgcttatatgggacagtgcttcaggtttaaaacagaacgctattccattagattctcaatgggttctttcctgcgctatttcgccatcgagtactttggtagcaagcgcaggattaaacaataactgtaccatttatagagtttcgaaagaaaacagagtagcgcaaaacgttgcgtcaattttcaaaggacatacttgctatatttctgacattgaatttacagataacgcacatatattgacagcaagtggggatatgacatgtgccttgtgggatataccgaaagcaaagagggtgagagaatattctgaccatttaggtgatgttttggcattagctattcctgaagagccaaacttagaaaattcttcgaacacattcgctagctgtggatcagacgggtatacttacatatgggatagcagatctccgtccgctgtacaaagcttttacgttaacgatagtgatattaatgcacttcgttttttcaaagacgggatgtcgattgttgcaggaagtgacaatggtgcgataaatatgtatgatttaaggtcggactgttctattgctactttttctctttttcgaggttatgaagaacgtacccctacccctacttatatggcagctaacatggagtacaataccgcgcaatcgccacaaactttaaaatcaacaagctcaagctatctagacaaccaaggcgttgtttctttagattttagtgcatctggaagattgatgtactcatgctatacagacattggttgtgttgtgtgggatgtattaaaaggagagattgttggaaaattagaaggtcatggtggcagagtcactggtgtgcgctcgagtccagatgggttagctgtatgtacaggttcatgggactcaaccatgaaaatatggtctccaggttatcaatagcttcgaattggaaatactgtgagcagtaattatcaatggatgctattatataaatatacatacctacacccatcccatatttacatagaataacaacagtaacattagttctgtggaagcgcaaaaacgtcctttaataaagtaagtcaaaacattcaacaatgaaaattcaaagcattgtcatttgcttcctttttctctttgggataaacgaaacaaaaacgaacaaaatgtcatgcactcaaaaattcttttcaatcgttttggaaacagtattattcactgacttatttgaccaacttgctagaatcatctatgttttcaggcattgtttaatttcatgatggctgtccctactttagcttgttatgagccttcactggctcgtccttatgtattgcgtctgacccaaaatttgtcctttcttgtttagtggaatttttgttcggtaatttcaaaaatgctgaattttcattaacaaatcatctggtagttgtgttataaacataaaaaactgctcccttctgggatgattttcaattgctctctgtactgcag

SEQ ID NO: 31 - protein, 423 aa, Saccharomyces cerevisiae
MAAHQMDSITYSNNVTQQYIQPQSLQDISAVEEEIQNKIEAARQESKQLHAQINKAKHKIQDASLFQMANKVTSLTKNKINLKPNIVLKGHNNKISDFRWSRDSKRILSASQDGFMLIWDSASGLKQNAIPLDSQWVLSCAISPSSTLVASAGLNNNCTIYRVSKENRVAQNVASIFKGHTCYISDIEFTDNAHILTASGDMTCALWDIPKAKRVREYSDHLGDVLALAIPEEPNLENSSNTFASCGSDGYTYIWDSRSPSAVQSFYVNDSDINALRFFKDGMSIVAGSDNGAINMYDLRSDCSIATFSLFRGYEERTPTPTYMAANMEYNTAQSPQTLKSTSSSYLDNQGVVSLDFSASGRLMYSCYTDIGCVVWDVLKGEIVGKLEGHGGRVTGVRSSPDGLAVCTGSWDSTMKIWSPGYQ